SEQUENCE LISTING (30 sequences)

SEQ ID NO: 1 - protein, 343 aa, Papaver somniferum
  REGION 1..343: note = N-terminaler Histidin-tag; Mittels Sequenzierung bestimmte Aminosaeuresequenz
MAHHHHHHSAALEVLFQGPGYQMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 2 - DNA, 1029 bp, Papaver somniferum
  misc_feature 1..1029: note = N-terminaler Histidin-tag; Fuer Sequenz-ID Nr. 1 kodierende Sequenz
  source 1..1029: mol_type = genomic DNA
atggcacatcaccaccaccatcactccgcggctcttgaagtcctctttcagggacccgggtaccagatggagagtagtggtgtaccagtaatcactctgggctcgggcaaggtgatgcctgttctgggcatgggaacatttgagaaagttggtaaagggtccgaaagagagaggttggcgattttaaaagcgatagaggtgggttacagatacttcgatacagctgctgcatacgaaactgaagaggttcttggagaagctattgctgaagcacttcaacttggcctagtcaaatctcgagatgaacttttcatcagttccatgctctggtgcactgatgctcacgctgatcgtgtcctcctcgctcttcagaattcgctgaggaatcttaaattggagtatgtggatctatatatgttacccttcccggcaagcttgaagcctgggaagataacgatggacataccagaggaagatatttgtcgcatggactacaggtctgtatgggcagccatggaagagtgtcaaaaccttggcttcactaaatcaatcggtgttagcaatttctcctgcaaaaagcttcaggaattgatggcgaccgccaacatccctccagctgtgaatcaagtggagatgagcccggctttccaacaaaagaagctgagagagtattgcaacgcaaataatatattagtcagtgcaatctctgtactgggatcaaacggaaccccatggggctccaatgcagttttgggttctgaggtgcttaagaaaattgctatggccaaaggaaaatctgttgctcaggttagtatgagatgggtttacgagcaaggcgcgagtcttgtggtaaaaagtttcagtgaagagagattgagggaaaacttgaacatatttgactgggaactcactaaggaagaccatgaaaagatcggtgagattccacagtgcagaatcttgagtgcttattttttggtctcacctaatggacctttcaaatctcaagaagagttgtgggatgatgaagct

SEQ ID NO: 3 - protein, 325 aa, Streptomyces aurantiacus
  REGION 1..325: note = N-terminaler Histidin-tag; N-terminale thrombin recognition and cleavage site; Codon-optimiert fuer E. coli; Mittels Sequenzierung bestimmte Aminosaeuresequenz
MGSSHHHHHHSSGLVPRGSHMATTTNSTNPTTASTLTPVTVIGLGAMGQALAGAFLKAGHPTTIWNRSPGKGEDLVARGATRAATPAEAVRAGEVVVVCVVDYEASQSILEPIAADLAGRVLVNVTSDAPERAREAGEWAAEHDIAYLDGAVMIPTVMIGTPDALLFYSGDKAAYDKHEGLLKSLGGQSAYVGADHGLAAVYDLSMLDFMFTSMSGLVHGYALAAKDGVPAASIAPFLKSHISLLSLLVEETAKNLDEGAYPGAEANLAMEVEGIEHILHAAERRGLDVSVLRGVRDVAQRAVDLGHGADSWSATVEGARNPAKL

SEQ ID NO: 4 - DNA, 975 bp, Streptomyces aurantiacus
  misc_feature 1..975: note = N-terminaler Histidin-tag; N-terminale thrombin recognition and cleavage site; Codon-optimiert fuer E. coli; Fuer Sequenz-ID Nr. 3 kodierende Sequenz
  source 1..975: mol_type = genomic DNA
atgggcagcagccatcatcatcatcatcacagcagcggcctggtgccgcgcggcagccatatggcaaccaccaccaatagtaccaatccgaccaccgcaagtaccctgaccccggtgaccgttattggtctgggtgccatgggccaggccctggcaggtgcatttctgaaagcaggtcatccgaccaccatttggaatcgtagcccgggtaaaggtgaagatctggtggcacgtggtgcaacccgcgccgcaacccctgcagaagcagtgcgtgcaggcgaagttgtggttgtgtgcgttgttgattatgaagccagccagagcattctggaaccgattgcagccgatctggcaggccgcgtgctggtgaatgtgaccagtgatgcaccggaacgcgcccgtgaagcaggtgaatgggccgccgaacatgatattgcctatctggatggtgcagttatgattccgaccgtgatgattggtaccccggatgcactgctgttttatagcggtgacaaagccgcatacgataaacatgaaggcctgctgaaaagcctgggtggccagagcgcatacgttggcgccgatcatggcctggccgcagtgtatgatctgagcatgctggattttatgtttaccagcatgagtggtctggttcatggctatgccctggccgccaaagatggcgtgccggcagccagtattgcaccgtttctgaaaagtcatattagcctgctgagcctgctggtggaagaaaccgccaaaaatctggatgaaggcgcatatccgggcgcagaagccaatctggcaatggaagtggaaggtattgaacatattctgcatgccgccgaacgccgtggtctggatgttagtgtgctgcgtggtgtgcgcgatgttgcccagcgcgccgtggatctgggtcatggtgccgatagttggagcgcaaccgttgaaggtgcacgcaatccggccaagctt

SEQ ID NO: 5 - protein, 737 aa, Arabidopsis thaliana
  REGION 1..737: note = N-terminales Flanking MG ; C-terminaler S-tag; Codon-optimiert fuer E. coli; Mittels Sequenzierung bestimmte Aminosaeuresequenz
MGMSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVWLESGKETAAAKFERQHMDSSTSAA

SEQ ID NO: 6 - DNA, 2211 bp, Arabidopsis thaliana
  misc_feature 1..2211: note = N-terminales Flanking MG ; C-terminaler S-tag; Codon-optimiert fuer E. coli; Fuer Sequenz-ID Nr. 5 kodierende Sequenz
  source 1..2211: mol_type = genomic DNA
atgggaatgagtagcagcagtagtagtagtaccagcatgattgatctgatggccgcaattattaagggtgaaccggttattgtgagcgatccggccaatgccagtgcctatgaaagtgtggccgcagaactgagtagcatgctgattgaaaatcgccagtttgcaatgattgtgaccaccagtattgcagtgctgattggctgtattgttatgctggtttggcgtcgtagtggtagcggcaatagtaaacgtgttgaaccgctgaaaccgctggttattaagccgcgcgaagaagaaattgatgatggtcgcaaaaaagtgaccattttctttggtacccagaccggcaccgccgaaggctttgcaaaagcactgggtgaagaagcaaaagcacgttatgaaaaaacccgctttaaaattgtggatctggatgattatgcagccgatgatgatgaatatgaagaaaaactgaagaaggaagatgtggcctttttctttctggccacctatggtgacggtgaaccgaccgataatgccgcccgcttttataaatggtttaccgaaggtaatgaccgtggtgaatggctgaaaaatctgaaatatggtgtgtttggtctgggtaatcgccagtatgaacattttaataaggtggcaaaagtggttgatgatattctggtggaacagggcgcccagcgcctggttcaggttggcctgggcgatgatgatcagtgcattgaagatgattttaccgcctggcgcgaagcactgtggccggaactggataccattctgcgcgaagaaggtgacaccgcagtggcaaccccgtataccgcagccgttctggaatatcgtgtgagcattcatgatagtgaagatgccaaattcaatgatattaacatggcaaatggcaatggttataccgtttttgatgcacagcatccgtataaagccaatgtggccgttaaacgtgaactgcataccccggaaagtgatcgtagctgcattcatctggaatttgatattgcaggtagtggcctgacctatgaaaccggtgaccatgtgggtgttctgtgtgataatctgagtgaaaccgtggatgaagcactgcgcctgctggatatgagcccggatacctattttagcctgcatgccgaaaaagaagatggcaccccgattagcagtagtctgccgccgccgtttccgccgtgcaatctgcgcaccgcactgacccgttatgcatgcctgctgagcagcccgaaaaaatcagccctggtggccctggcagcccatgcctcagatccgaccgaagccgaacgcctgaaacatctggcaagtccggccggcaaagatgaatatagcaaatgggtggttgaaagccagcgcagcctgctggaagtgatggccgaatttccgagcgcaaaaccgccgctgggcgttttctttgcaggcgttgccccgcgtctgcagccgagattttatagcattagtagcagcccgaagattgcagaaacccgcattcatgtgacctgtgcactggtgtatgaaaaaatgccgaccggtcgtattcataaaggtgtttgtagtacctggatgaaaaatgcagtgccgtatgaaaaaagcgaaaattgtagcagcgcaccgatttttgtgcgccagagcaattttaaactgccgagcgatagcaaagttccgattattatgattggtccgggtaccggtctggcaccgtttcgtggctttctgcaggaacgcctggccctggtggaaagtggtgtggaactgggcccgagtgtgctgtttttcggctgccgcaatcgccgtatggattttatctatgaagaagaactgcagcgctttgttgaaagcggtgccctggccgaactgagtgttgcctttagtcgcgaaggtccgaccaaagaatatgttcagcataaaatgatggacaaagccagtgatatttggaatatgattagccagggtgcctatctgtatgtttgcggcgatgccaaaggtatggcacgtgatgtgcatcgcagtctgcataccattgcacaggaacagggcagcatggatagtaccaaagccgaaggttttgtgaaaaatctgcagaccagtggccgctatctgcgcgatgtgtggctcgagtctggtaaagaaaccgctgctgcgaaatttgaacgccagcacatggactcgtctactagcgcagct

SEQ ID NO: 7 - protein, 515 aa, Papaver somniferum
  REGION 1..515: note = N-terminales Flanking MG ; C-terminaler His-tag; Codon-optimiert fuer E. coli; Mittels Sequenzierung bestimmte Aminosaeuresequenz
MGMAPINIEGNDFWMIACTVIIVFALVKFMFSKISFYQSANTTEWPAGPKTLPIIGNLHQLGGGVPLQVALANLAKVYGGAFTIWIGSWVPMIVISDIDNAREVLVNKSADYSARDVPDILKIITANGKNIADCDSGPFWHNLKKGLQSCINPSNVMSLSRLQEKDMQNLIKSMQERASQHNGIIKPLDHAKEASMRLLSRVIFGHDFSNEDLVIGVKDALDEMVRISGLASLADAFKIAKYLPSQRKNIRDMYATRDRVYNLIQPHIVPNLPENSFLHFLTSQDYSDEIIYSMVLEIFGLGVDSTAATAVWALSFLVGEQEIQEKLYREINNRTGGQRPVKVVDLKELPYLQAVMKETLRMKPIAPLAVPHVAAKDTTFKGRRIVKGTKVMVNLYAIHHDPNVFPAPYKFMPERFLKDVNSDGRFGDINTMESSLIPFGAGMRICGGVELAKQMVAFALASMVNEFKWDCVSEGKLPDLSEAISFILYMKNPLEAKITPRTKPFRQLEHHHHHH

SEQ ID NO: 8 - DNA, 1545 bp, Papaver somniferum
  misc_feature 1..1545: note = N-terminales Flanking MG ; C-terminaler His-tag; Codon-optimiert fuer E. coli; Fuer Sequenz-ID Nr. 7 kodierende Sequenz
  source 1..1545: mol_type = genomic DNA
atgggaatggcccctattaatattgaaggtaatgatttttggatgatcgcatgcaccgtgattattgtgtttgcactggttaaattcatgtttagtaagattagcttctaccagagtgcaaataccaccgaatggccggccggtccgaaaaccctgccgattattggcaatctgcatcagctgggcggtggcgtgccgctgcaggttgctctggccaatctggcaaaagtttatggcggcgcctttaccatttggattggtagttgggttccgatgattgttattagtgatattgataacgcgcgtgaagtgctggttaataagagcgcagattatagtgcccgtgatgtgccggatattctgaaaattattaccgccaatggcaaaaatatcgcagattgcgatagcggtccgttttggcataatctgaaaaaaggcctgcagagctgcattaatccgagtaatgtgatgagcctgagtcgcctgcaggaaaaagatatgcagaatctgattaagagtatgcaggaacgtgcaagtcagcataatggtattattaagccgctggatcatgcaaaagaagcaagcatgcgcctgctgagtcgcgtgatttttggtcatgattttagtaatgaggacctggttattggtgttaaagatgcactggatgaaatggttcgtattagtggcctggccagcctggccgatgcctttaaaattgcaaaatatctgccgagtcagcgcaaaaatattcgcgatatgtatgccacccgtgatcgtgtttataatctgattcagccgcatattgttccgaatctgccggaaaatagctttctgcattttctgaccagccaggattatagcgatgaaattatctatagtatggtgctggaaattttcggtctgggcgtggatagtaccgcagcaaccgcagtgtgggccctgagttttctggttggcgaacaggaaattcaggaaaaactgtatcgtgaaattaacaatcgtaccggtggtcagcgcccggttaaagttgttgatctgaaagaactgccgtatctgcaggccgttatgaaagaaaccctgcgcatgaaaccgattgcaccgctggccgttccgcatgtggccgcaaaagataccacctttaaaggccgtcgcattgttaaaggcaccaaagttatggtgaatctgtatgcaattcatcatgatccgaatgtgtttccggcaccgtataaattcatgccggaacgttttctgaaagatgttaatagtgatggtcgttttggtgacattaatacaatggaaagcagcctgattccgtttggtgcaggtatgcgcatttgtggcggtgttgaactggccaaacagatggtggcatttgccctggcaagcatggttaatgaattcaaatgggattgcgttagcgaaggtaaactgccggatctgagtgaagccattagctttattctgtatatgaaaaacccgctggaagcaaaaattaccccgcgtaccaaaccgtttcgccagctcgagcaccaccaccaccaccac

SEQ ID NO: 9 - protein, 486 aa, Papaver somniferum
  REGION 1..486: note = N-terminales Entfernen der Transmembrandomaene (29 Aminosaeuren) und N-terminales Flanking MG ; C-terminaler His-tag; Codon-optimiert fuer E. coli; Mittels Sequenzierung bestimmte Aminosaeuresequenz
MGMKISFYQSANTTEWPAGPKTLPIIGNLHQLGGGVPLQVALANLAKVYGGAFTIWIGSWVPMIVISDIDNAREVLVNKSADYSARDVPDILKIITANGKNIADCDSGPFWHNLKKGLQSCINPSNVMSLSRLQEKDMQNLIKSMQERASQHNGIIKPLDHAKEASMRLLSRVIFGHDFSNEDLVIGVKDALDEMVRISGLASLADAFKIAKYLPSQRKNIRDMYATRDRVYNLIQPHIVPNLPENSFLHFLTSQDYSDEIIYSMVLEIFGLGVDSTAATAVWALSFLVGEQEIQEKLYREINNRTGGQRPVKVVDLKELPYLQAVMKETLRMKPIAPLAVPHVAAKDTTFKGRRIVKGTKVMVNLYAIHHDPNVFPAPYKFMPERFLKDVNSDGRFGDINTMESSLIPFGAGMRICGGVELAKQMVAFALASMVNEFKWDCVSEGKLPDLSEAISFILYMKNPLEAKITPRTKPFRQLEHHHHHH

SEQ ID NO: 10 - DNA, 1458 bp, Papaver somniferum
  misc_feature 1..1458: note = N-terminales Entfernen der Transmembrandomaene (29 Aminosaeuren) und N-terminales Flanking MG ; C-terminaler His-tag; Codon-optimiert fuer E. coli; Fuer Sequenz-ID Nr. 9 kodierende Sequenz
  source 1..1458: mol_type = genomic DNA
atgggaatgaagatcagtttctaccagagcgcaaataccaccgaatggccggccggtccgaaaaccctgccgattattggtaatctgcatcagctgggcggcggcgttccgctgcaggttgctctggcaaatctggccaaagtttatggcggtgcctttaccatttggattggtagctgggttccgatgattgttattagtgatattgataacgcccgtgaagttctggttaataagagcgcagattatagcgcacgcgatgtgccggatattctgaaaattattaccgccaatggtaaaaatatcgccgattgcgatagcggcccgttttggcataatctgaaaaaaggcctgcagagctgcattaatccgagtaatgttatgagcctgagtcgcctgcaggaaaaagatatgcagaatctgattaagagcatgcaggaacgcgcaagccagcataatggcattattaagccgctggatcatgcaaaagaagcaagcatgcgtctgctgagtcgtgtgatttttggccatgattttagcaatgaagatctggttattggcgtgaaagatgccctggatgaaatggttcgtattagcggcctggcaagcctggcagatgcattcaaaattgccaaatatctgccgagtcagcgcaaaaatattcgtgatatgtatgccacccgtgatcgtgtgtataatctgattcagccgcatattgttccgaatctgccggaaaatagttttctgcattttctgaccagccaggattatagtgatgaaattatctatagcatggtgctggaaatttttggtctgggtgttgatagtaccgcagcaaccgcagtgtgggccctgagttttctggtgggcgaacaggaaattcaggaaaaactgtatcgcgaaattaataatcgcaccggtggtcagcgtccggttaaagttgtggatctgaaagaactgccgtatctgcaggcagtgatgaaagaaaccctgcgcatgaaaccgattgcaccgctggccgttccgcatgtggccgcaaaagataccacctttaaaggtcgccgcattgttaaaggcaccaaagtgatggttaatctgtatgccattcatcatgatccgaatgtttttccggcaccgtataaattcatgccggaacgttttctgaaagatgtgaatagcgatggccgctttggcgatattaatacaatggaaagcagcctgattccgtttggtgccggtatgcgtatttgtggcggtgtggaactggcaaaacagatggttgcatttgcactggccagtatggttaatgaattcaaatgggattgtgtgagcgaaggcaaactgccggatctgagtgaagccattagctttattctgtatatgaaaaacccgctggaagccaaaattaccccgcgcaccaaaccgtttcgtcagctcgagcaccaccaccaccaccac

SEQ ID NO: 11 - protein, 504 aa, Papaver somniferum
  REGION 1..504: note = N-terminales Flanking MG ; C-terminaler His-tag, Codon-optimiert fuer E. coli; Mittels Sequenzierung bestimmte Aminosaeuresequenz
MGMEESLWVVTATVVVVFAIAKLLKKSSSISTMEWPKGPKKLPIIGNLHQLGGEAFHVVLANLAKIHGTVMTIWVGAWRPMIVISDIDNAREVLVNKSADYSARDVPDILKIITANGKNIADCDSGPFWHNLKKGLQSCINPSNVMSLSRLQEKDMQNLIKSMQERASQHNGIIKPLDHAKEASMRLLSRVIFGHDFSNEDLVIGVKDALDEMVRISGLASLADAFKIAKYLPSQRKNIRDMYATRDRVYNLIQPHIVPNLPANSFLYFLTSQDYSDEIIYSMVLEIFGLGVDSTAATAVWALSFLVGEQEIQEKLYREINNRTGGQRPVKVVDLKELPYLQAVMKETLRMKPIAPLAVPHVAAKDTTFKGRRIVKGTKVMVNLYAIHHDPNVFPAPYKFMPERFLKDVNSDGRFGDINTMESSLIPFGAGMRICGGVELAKQMVAFALASMVNEFKWDCVSEGKLPDLSEAISFILYMKNPLEAKITPRTKPFRQLEHHHHHH

SEQ ID NO: 12 - DNA, 1512 bp, Papaver somniferum
  misc_feature 1..1512: note = N-terminales Flanking MG ; C-terminaler His-tag, Codon-optimiert fuer E. coli; Fuer Sequenz-ID Nr. 11 kodierende Sequenz
  source 1..1512: mol_type = genomic DNA
atgggaatggaagaaagtctgtgggttgttaccgccaccgtggttgtggtttttgccattgcaaaactgctgaaaaaatctagcagtattagcacaatggaatggccgaaaggcccgaaaaaactgccgattattggcaatctgcatcagctgggcggtgaagcctttcatgtggttctggccaatctggcaaaaattcatggcaccgttatgaccatttgggtgggtgcctggcgtccgatgattgtgattagcgatattgataatgcccgtgaagtgctggttaataagagcgcagattatagcgcccgcgatgttccggatattctgaaaattattaccgcaaatggtaaaaacatcgccgattgtgatagcggcccgttttggcataatctgaaaaaaggtctgcagagttgcattaatccgagtaatgtgatgagcctgagtcgtctgcaggaaaaagatatgcagaatctgattaagagcatgcaggaacgtgcaagccagcataatggcattattaagccgctggatcatgccaaagaagccagtatgcgtctgctgagccgtgtgatttttggccatgattttagcaatgaagatctggttattggcgttaaagatgccctggatgaaatggttcgcattagcggtctggccagcctggcagatgcattcaaaattgcaaaatatctgccgagccagcgcaaaaatattcgcgatatgtatgccacccgcgatcgtgtgtataatctgattcagccgcatattgttccgaatctgccggccaatagttttctgtattttctgaccagtcaggattatagcgatgaaattatctatagtatggtgctggaaattttcggcctgggtgtggatagcaccgcagcaaccgcagtttgggccctgagttttctggtgggtgaacaggaaattcaggaaaaactgtatcgcgaaattaataatcgtaccggtggtcagcgtccggttaaagtggtggatctgaaagaactgccgtatctgcaggcagtgatgaaagaaaccctgcgcatgaaaccgattgccccgctggccgtgccgcatgttgcagcaaaagataccacctttaaaggtcgccgcattgttaaaggtaccaaagtgatggttaatctgtatgcaattcatcatgatccgaatgtttttccggcaccgtataaattcatgccggaacgctttctgaaagatgttaatagcgatggccgctttggtgacattaatacaatggaaagcagcctgattccgtttggtgccggtatgcgcatttgcggcggcgtggaactggcaaaacagatggttgcctttgccctggccagtatggtgaatgaattcaaatgggattgtgtgagtgaaggcaaactgccggatctgagcgaagcaattagctttattctgtatatgaagaacccgctggaagcaaaaattaccccgcgcaccaaaccgtttcgccagctcgagcaccaccaccaccaccac

SEQ ID NO: 13 - protein, 771 aa, Papaver somniferum
  REGION 1..771: note = N-terminaler His-tag; N-terminaler GST-tag; Codon-optimiert fuer E. coli; Mittels Sequenzierung bestimmte Aminosaeuresequenz
MGSSHHHHHHGTSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGTDYDIPTTENLYFQGHMASMTGGQQMGRGSEFELMAPINIEGNDFWMIACTVIIVFALVKFMFSKISFYQSANTTEWPAGPKTLPIIGNLHQLGGGVPLQVALANLAKVYGGAFTIWIGSWVPMIVISDIDNAREVLVNKSADYSARDVPDILKIITANGKNIADCDSGPFWHNLKKGLQSCINPSNVMSLSRLQEKDMQNLIKSMQERASQHNGIIKPLDHAKEASMRLLSRVIFGHDFSNEDLVIGVKDALDEMVRISGLASLADAFKIAKYLPSQRKNIRDMYATRDRVYNLIQPHIVPNLPENSFLHFLTSQDYSDEIIYSMVLEIFGLGVDSTAATAVWALSFLVGEQEIQEKLYREINNRTGGQRPVKVVDLKELPYLQAVMKETLRMKPIAPLAVPHVAAKDTTFKGRRIVKGTKVMVNLYAIHHDPNVFPAPYKFMPERFLKDVNSDGRFGDINTMESSLIPFGAGMRICGGVELAKQMVAFALASMVNEFKWDCVSEGKLPDLSEAISFILYMKNPLEAKITPRTKPFRQ

SEQ ID NO: 14 - DNA, 2313 bp, Papaver somniferum
  misc_feature 1..2313: note = N-terminaler His-tag; N-terminaler GST-tag; Codon-optimiert fuer E. coli; Fuer Sequenz-ID Nr. 13 kodierende Sequenz
  source 1..2313: mol_type = genomic DNA
atgggcagcagccatcatcatcatcatcacggtacctcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatggtaccgattacgatatcccaacgaccgaaaacctttacttccagggccatatggctagcatgactggtggacagcaaatgggtcgcggatccgaattcgagctcatggcccctattaatattgaaggtaatgatttttggatgatcgcatgcaccgtgattattgtgtttgcactggttaaattcatgtttagtaagattagcttctaccagagtgcaaataccaccgaatggccggccggtccgaaaaccctgccgattattggcaatctgcatcagctgggcggtggcgtgccgctgcaggttgctctggccaatctggcaaaagtttatggcggcgcctttaccatttggattggtagttgggttccgatgattgttattagtgatattgataacgcgcgtgaagtgctggttaataagagcgcagattatagtgcccgtgatgtgccggatattctgaaaattattaccgccaatggcaaaaatatcgcagattgcgatagcggtccgttttggcataatctgaaaaaaggcctgcagagctgcattaatccgagtaatgtgatgagcctgagtcgcctgcaggaaaaagatatgcagaatctgattaagagtatgcaggaacgtgcaagtcagcataatggtattattaagccgctggatcatgcaaaagaagcaagcatgcgcctgctgagtcgcgtgatttttggtcatgattttagtaatgaggacctggttattggtgttaaagatgcactggatgaaatggttcgtattagtggcctggccagcctggccgatgcctttaaaattgcaaaatatctgccgagtcagcgcaaaaatattcgcgatatgtatgccacccgtgatcgtgtttataatctgattcagccgcatattgttccgaatctgccggaaaatagctttctgcattttctgaccagccaggattatagcgatgaaattatctatagtatggtgctggaaattttcggtctgggcgtggatagtaccgcagcaaccgcagtgtgggccctgagttttctggttggcgaacaggaaattcaggaaaaactgtatcgtgaaattaacaatcgtaccggtggtcagcgcccggttaaagttgttgatctgaaagaactgccgtatctgcaggccgttatgaaagaaaccctgcgcatgaaaccgattgcaccgctggccgttccgcatgtggccgcaaaagataccacctttaaaggccgtcgcattgttaaaggcaccaaagttatggtgaatctgtatgcaattcatcatgatccgaatgtgtttccggcaccgtataaattcatgccggaacgttttctgaaagatgttaatagtgatggtcgttttggtgacattaatacaatggaaagcagcctgattccgtttggtgcaggtatgcgcatttgtggcggtgttgaactggccaaacagatggtggcatttgccctggcaagcatggttaatgaattcaaatgggattgcgttagcgaaggtaaactgccggatctgagtgaagccattagctttattctgtatatgaaaaacccgctggaagcaaaaattaccccgcgtaccaaaccgtttcgccag

SEQ ID NO: 15 - protein, 742 aa, Papaver somniferum
  REGION 1..742: note = N-terminales Entfernen der Transmembrandomaene (29 Aminosaeuren); N-terminaler His-tag; N-terminaler GST-tag; Codon-optimiert fuer E. coli; Mittels Sequenzierung bestimmte Aminosaeuresequenz
MGSSHHHHHHGTSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGTDYDIPTTENLYFQGHMASMTGGQQMGRGSEFELMKISFYQSANTTEWPAGPKTLPIIGNLHQLGGGVPLQVALANLAKVYGGAFTIWIGSWVPMIVISDIDNAREVLVNKSADYSARDVPDILKIITANGKNIADCDSGPFWHNLKKGLQSCINPSNVMSLSRLQEKDMQNLIKSMQERASQHNGIIKPLDHAKEASMRLLSRVIFGHDFSNEDLVIGVKDALDEMVRISGLASLADAFKIAKYLPSQRKNIRDMYATRDRVYNLIQPHIVPNLPENSFLHFLTSQDYSDEIIYSMVLEIFGLGVDSTAATAVWALSFLVGEQEIQEKLYREINNRTGGQRPVKVVDLKELPYLQAVMKETLRMKPIAPLAVPHVAAKDTTFKGRRIVKGTKVMVNLYAIHHDPNVFPAPYKFMPERFLKDVNSDGRFGDINTMESSLIPFGAGMRICGGVELAKQMVAFALASMVNEFKWDCVSEGKLPDLSEAISFILYMKNPLEAKITPRTKPFRQ

SEQ ID NO: 16 - DNA, 2226 bp, Papaver somniferum
  misc_feature 1..2226: note = N-terminales Entfernen der Transmembrandomaene (29 Aminosaeuren); N-terminaler His-tag; N-terminaler GST-tag; Codon-optimiert fuer E. coli; Fuer Sequenz-ID Nr. 15 kodierende Sequenz
  source 1..2226: mol_type = genomic DNA
atgggcagcagccatcatcatcatcatcacggtacctcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatggtaccgattacgatatcccaacgaccgaaaacctttacttccagggccatatggctagcatgactggtggacagcaaatgggtcgcggatccgaattcgagctcatgaagatcagtttctaccagagcgcaaataccaccgaatggccggccggtccgaaaaccctgccgattattggtaatctgcatcagctgggcggcggcgttccgctgcaggttgctctggcaaatctggccaaagtttatggcggtgcctttaccatttggattggtagctgggttccgatgattgttattagtgatattgataacgcccgtgaagttctggttaataagagcgcagattatagcgcacgcgatgtgccggatattctgaaaattattaccgccaatggtaaaaatatcgccgattgcgatagcggcccgttttggcataatctgaaaaaaggcctgcagagctgcattaatccgagtaatgttatgagcctgagtcgcctgcaggaaaaagatatgcagaatctgattaagagcatgcaggaacgcgcaagccagcataatggcattattaagccgctggatcatgcaaaagaagcaagcatgcgtctgctgagtcgtgtgatttttggccatgattttagcaatgaagatctggttattggcgtgaaagatgccctggatgaaatggttcgtattagcggcctggcaagcctggcagatgcattcaaaattgccaaatatctgccgagtcagcgcaaaaatattcgtgatatgtatgccacccgtgatcgtgtgtataatctgattcagccgcatattgttccgaatctgccggaaaatagttttctgcattttctgaccagccaggattatagtgatgaaattatctatagcatggtgctggaaatttttggtctgggtgttgatagtaccgcagcaaccgcagtgtgggccctgagttttctggtgggcgaacaggaaattcaggaaaaactgtatcgcgaaattaataatcgcaccggtggtcagcgtccggttaaagttgtggatctgaaagaactgccgtatctgcaggcagtgatgaaagaaaccctgcgcatgaaaccgattgcaccgctggccgttccgcatgtggccgcaaaagataccacctttaaaggtcgccgcattgttaaaggcaccaaagtgatggttaatctgtatgccattcatcatgatccgaatgtttttccggcaccgtataaattcatgccggaacgttttctgaaagatgtgaatagcgatggccgctttggcgatattaatacaatggaaagcagcctgattccgtttggtgccggtatgcgtatttgtggcggtgtggaactggcaaaacagatggttgcatttgcactggccagtatggttaatgaattcaaatgggattgtgtgagcgaaggcaaactgccggatctgagtgaagccattagctttattctgtatatgaaaaacccgctggaagccaaaattaccccgcgcaccaaaccgtttcgtcag

SEQ ID NO: 17 - protein, 633 aa, Papaver somniferum
  REGION 1..633: note = N-terminaler His-tag; N-terminaler SUMO-tag; Codon-optimiert fuer E. coli; Mittels Sequenzierung bestimmte Aminosaeuresequenz
MGSSHHHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGRGSEFELMAPINIEGNDFWMIACTVIIVFALVKFMFSKISFYQSANTTEWPAGPKTLPIIGNLHQLGGGVPLQVALANLAKVYGGAFTIWIGSWVPMIVISDIDNAREVLVNKSADYSARDVPDILKIITANGKNIADCDSGPFWHNLKKGLQSCINPSNVMSLSRLQEKDMQNLIKSMQERASQHNGIIKPLDHAKEASMRLLSRVIFGHDFSNEDLVIGVKDALDEMVRISGLASLADAFKIAKYLPSQRKNIRDMYATRDRVYNLIQPHIVPNLPENSFLHFLTSQDYSDEIIYSMVLEIFGLGVDSTAATAVWALSFLVGEQEIQEKLYREINNRTGGQRPVKVVDLKELPYLQAVMKETLRMKPIAPLAVPHVAAKDTTFKGRRIVKGTKVMVNLYAIHHDPNVFPAPYKFMPERFLKDVNSDGRFGDINTMESSLIPFGAGMRICGGVELAKQMVAFALASMVNEFKWDCVSEGKLPDLSEAISFILYMKNPLEAKITPRTKPFRQ

SEQ ID NO: 18 - DNA, 1899 bp, Papaver somniferum
  misc_feature 1..1899: note = N-terminaler His-tag; N-terminaler SUMO-tag; Codon-optimiert fuer E. coli; Fuer Sequenz-ID Nr. 17 kodierende Sequenz
  source 1..1899: mol_type = genomic DNA
atgggcagcagccatcaccatcatcatcatcatcacggcagcggcctggtgccgcgcggcagcgctagcatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaatacaagctgatcagacccctgaagatttggacatggaggataacgatattattgaggctcacagagaacagattggtggtcgcggatccgaattcgagctcatggcccctattaatattgaaggtaatgatttttggatgatcgcatgcaccgtgattattgtgtttgcactggttaaattcatgtttagtaagattagcttctaccagagtgcaaataccaccgaatggccggccggtccgaaaaccctgccgattattggcaatctgcatcagctgggcggtggcgtgccgctgcaggttgctctggccaatctggcaaaagtttatggcggcgcctttaccatttggattggtagttgggttccgatgattgttattagtgatattgataacgcgcgtgaagtgctggttaataagagcgcagattatagtgcccgtgatgtgccggatattctgaaaattattaccgccaatggcaaaaatatcgcagattgcgatagcggtccgttttggcataatctgaaaaaaggcctgcagagctgcattaatccgagtaatgtgatgagcctgagtcgcctgcaggaaaaagatatgcagaatctgattaagagtatgcaggaacgtgcaagtcagcataatggtattattaagccgctggatcatgcaaaagaagcaagcatgcgcctgctgagtcgcgtgatttttggtcatgattttagtaatgaggacctggttattggtgttaaagatgcactggatgaaatggttcgtattagtggcctggccagcctggccgatgcctttaaaattgcaaaatatctgccgagtcagcgcaaaaatattcgcgatatgtatgccacccgtgatcgtgtttataatctgattcagccgcatattgttccgaatctgccggaaaatagctttctgcattttctgaccagccaggattatagcgatgaaattatctatagtatggtgctggaaattttcggtctgggcgtggatagtaccgcagcaaccgcagtgtgggccctgagttttctggttggcgaacaggaaattcaggaaaaactgtatcgtgaaattaacaatcgtaccggtggtcagcgcccggttaaagttgttgatctgaaagaactgccgtatctgcaggccgttatgaaagaaaccctgcgcatgaaaccgattgcaccgctggccgttccgcatgtggccgcaaaagataccacctttaaaggccgtcgcattgttaaaggcaccaaagttatggtgaatctgtatgcaattcatcatgatccgaatgtgtttccggcaccgtataaattcatgccggaacgttttctgaaagatgttaatagtgatggtcgttttggtgacattaatacaatggaaagcagcctgattccgtttggtgcaggtatgcgcatttgtggcggtgttgaactggccaaacagatggtggcatttgccctggcaagcatggttaatgaattcaaatgggattgcgttagcgaaggtaaactgccggatctgagtgaagccattagctttattctgtatatgaaaaacccgctggaagcaaaaattaccccgcgtaccaaaccgtttcgccag

SEQ ID NO: 19 - protein, 604 aa, Papaver somniferum
  REGION 1..604: note = N-terminales Entfernen der Transmembrandomaene (29 Aminosaeuren); N-terminaler His-tag; N-terminaler SUMO-tag; Codon-optimiert fuer E. coli; Mittels Sequenzierung bestimmte Aminosaeuresequenz
MGSSHHHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGRGSEFELMKISFYQSANTTEWPAGPKTLPIIGNLHQLGGGVPLQVALANLAKVYGGAFTIWIGSWVPMIVISDIDNAREVLVNKSADYSARDVPDILKIITANGKNIADCDSGPFWHNLKKGLQSCINPSNVMSLSRLQEKDMQNLIKSMQERASQHNGIIKPLDHAKEASMRLLSRVIFGHDFSNEDLVIGVKDALDEMVRISGLASLADAFKIAKYLPSQRKNIRDMYATRDRVYNLIQPHIVPNLPENSFLHFLTSQDYSDEIIYSMVLEIFGLGVDSTAATAVWALSFLVGEQEIQEKLYREINNRTGGQRPVKVVDLKELPYLQAVMKETLRMKPIAPLAVPHVAAKDTTFKGRRIVKGTKVMVNLYAIHHDPNVFPAPYKFMPERFLKDVNSDGRFGDINTMESSLIPFGAGMRICGGVELAKQMVAFALASMVNEFKWDCVSEGKLPDLSEAISFILYMKNPLEAKITPRTKPFRQ

SEQ ID NO: 20 - DNA, 1812 bp, Papaver somniferum
  misc_feature 1..1812: note = N-terminales Entfernen der Transmembrandomaene (29 Aminosaeuren); N-terminaler His-tag; N-terminaler SUMO-tag; Codon-optimiert fuer E. coli; Fuer Sequenz-ID Nr. 19 kodierende Sequenz
  source 1..1812: mol_type = genomic DNA
atgggcagcagccatcaccatcatcatcatcatcacggcagcggcctggtgccgcgcggcagcgctagcatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaatacaagctgatcagacccctgaagatttggacatggaggataacgatattattgaggctcacagagaacagattggtggtcgcggatccgaattcgagctcatgaagatcagtttctaccagagcgcaaataccaccgaatggccggccggtccgaaaaccctgccgattattggtaatctgcatcagctgggcggcggcgttccgctgcaggttgctctggcaaatctggccaaagtttatggcggtgcctttaccatttggattggtagctgggttccgatgattgttattagtgatattgataacgcccgtgaagttctggttaataagagcgcagattatagcgcacgcgatgtgccggatattctgaaaattattaccgccaatggtaaaaatatcgccgattgcgatagcggcccgttttggcataatctgaaaaaaggcctgcagagctgcattaatccgagtaatgttatgagcctgagtcgcctgcaggaaaaagatatgcagaatctgattaagagcatgcaggaacgcgcaagccagcataatggcattattaagccgctggatcatgcaaaagaagcaagcatgcgtctgctgagtcgtgtgatttttggccatgattttagcaatgaagatctggttattggcgtgaaagatgccctggatgaaatggttcgtattagcggcctggcaagcctggcagatgcattcaaaattgccaaatatctgccgagtcagcgcaaaaatattcgtgatatgtatgccacccgtgatcgtgtgtataatctgattcagccgcatattgttccgaatctgccggaaaatagttttctgcattttctgaccagccaggattatagtgatgaaattatctatagcatggtgctggaaatttttggtctgggtgttgatagtaccgcagcaaccgcagtgtgggccctgagttttctggtgggcgaacaggaaattcaggaaaaactgtatcgcgaaattaataatcgcaccggtggtcagcgtccggttaaagttgtggatctgaaagaactgccgtatctgcaggcagtgatgaaagaaaccctgcgcatgaaaccgattgcaccgctggccgttccgcatgtggccgcaaaagataccacctttaaaggtcgccgcattgttaaaggcaccaaagtgatggttaatctgtatgccattcatcatgatccgaatgtttttccggcaccgtataaattcatgccggaacgttttctgaaagatgtgaatagcgatggccgctttggcgatattaatacaatggaaagcagcctgattccgtttggtgccggtatgcgtatttgtggcggtgtggaactggcaaaacagatggttgcatttgcactggccagtatggttaatgaattcaaatgggattgtgtgagcgaaggcaaactgccggatctgagtgaagccattagctttattctgtatatgaaaaacccgctggaagccaaaattaccccgcgcaccaaaccgtttcgtcag

SEQ ID NO: 21 - protein, 494 aa, Papaver somniferum
  REGION 1..494: note = N-terminales Austauschen der Transmembrandomaene (29 Aminosaeuren) mit der Sequenz ALLLAVFL und N-terminales Flanking MG ; C-terminaler His-tag; Codon-optimiert fuer E. coli; Mittels Sequenzierung bestimmte Aminosaeuresequenz
MGMALLLAVFLKISFYQSANTTEWPAGPKTLPIIGNLHQLGGGVPLQVALANLAKVYGGAFTIWIGSWVPMIVISDIDNAREVLVNKSADYSARDVPDILKIITANGKNIADCDSGPFWHNLKKGLQSCINPSNVMSLSRLQEKDMQNLIKSMQERASQHNGIIKPLDHAKEASMRLLSRVIFGHDFSNEDLVIGVKDALDEMVRISGLASLADAFKIAKYLPSQRKNIRDMYATRDRVYNLIQPHIVPNLPENSFLHFLTSQDYSDEIIYSMVLEIFGLGVDSTAATAVWALSFLVGEQEIQEKLYREINNRTGGQRPVKVVDLKELPYLQAVMKETLRMKPIAPLAVPHVAAKDTTFKGRRIVKGTKVMVNLYAIHHDPNVFPAPYKFMPERFLKDVNSDGRFGDINTMESSLIPFGAGMRICGGVELAKQMVAFALASMVNEFKWDCVSEGKLPDLSEAISFILYMKNPLEAKITPRTKPFRQLEHHHHHH

SEQ ID NO: 22 - DNA, 1482 bp, Papaver somniferum
  misc_feature 1..1482: note = N-terminales Austauschen der Transmembrandomaene (29 Aminosaeuren) mit der Sequenz ALLLAVFL und N-terminales Flanking MG ; C-terminaler His-tag; Codon-optimiert fuer E. coli; Fuer Sequenz-ID Nr. 21 kodierende Sequenz
  source 1..1482: mol_type = genomic DNA
atgggaatggcacttctgctggcagttttccttaagatcagtttctaccagagcgcaaataccaccgaatggccggccggtccgaaaaccctgccgattattggtaatctgcatcagctgggcggcggcgttccgctgcaggttgctctggcaaatctggccaaagtttatggcggtgcctttaccatttggattggtagctgggttccgatgattgttattagtgatattgataacgcccgtgaagttctggttaataagagcgcagattatagcgcacgcgatgtgccggatattctgaaaattattaccgccaatggtaaaaatatcgccgattgcgatagcggcccgttttggcataatctgaaaaaaggcctgcagagctgcattaatccgagtaatgttatgagcctgagtcgcctgcaggaaaaagatatgcagaatctgattaagagcatgcaggaacgcgcaagccagcataatggcattattaagccgctggatcatgcaaaagaagcaagcatgcgtctgctgagtcgtgtgatttttggccatgattttagcaatgaagatctggttattggcgtgaaagatgccctggatgaaatggttcgtattagcggcctggcaagcctggcagatgcattcaaaattgccaaatatctgccgagtcagcgcaaaaatattcgtgatatgtatgccacccgtgatcgtgtgtataatctgattcagccgcatattgttccgaatctgccggaaaatagttttctgcattttctgaccagccaggattatagtgatgaaattatctatagcatggtgctggaaatttttggtctgggtgttgatagtaccgcagcaaccgcagtgtgggccctgagttttctggtgggcgaacaggaaattcaggaaaaactgtatcgcgaaattaataatcgcaccggtggtcagcgtccggttaaagttgtggatctgaaagaactgccgtatctgcaggcagtgatgaaagaaaccctgcgcatgaaaccgattgcaccgctggccgttccgcatgtggccgcaaaagataccacctttaaaggtcgccgcattgttaaaggcaccaaagtgatggttaatctgtatgccattcatcatgatccgaatgtttttccggcaccgtataaattcatgccggaacgttttctgaaagatgtgaatagcgatggccgctttggcgatattaatacaatggaaagcagcctgattccgtttggtgccggtatgcgtatttgtggcggtgtggaactggcaaaacagatggttgcatttgcactggccagtatggttaatgaattcaaatgggattgtgtgagcgaaggcaaactgccggatctgagtgaagccattagctttattctgtatatgaaaaacccgctggaagccaaaattaccccgcgcaccaaaccgtttcgtcagctcgagcaccaccaccaccaccac

SEQ ID NO: 23 - protein, 492 aa, Papaver somniferum
  REGION 1..492: note = N-terminales Austauschen der Transmembrandomaene (29 Aminosaeuren) mit der Sequenz AKKTSS und N-terminales Flanking MG ; C-terminaler His-tag; Codon-optimiert fuer E. coli; Mittels Sequenzierung bestimmte Aminosaeuresequenz
MGMAKKTSSKISFYQSANTTEWPAGPKTLPIIGNLHQLGGGVPLQVALANLAKVYGGAFTIWIGSWVPMIVISDIDNAREVLVNKSADYSARDVPDILKIITANGKNIADCDSGPFWHNLKKGLQSCINPSNVMSLSRLQEKDMQNLIKSMQERASQHNGIIKPLDHAKEASMRLLSRVIFGHDFSNEDLVIGVKDALDEMVRISGLASLADAFKIAKYLPSQRKNIRDMYATRDRVYNLIQPHIVPNLPENSFLHFLTSQDYSDEIIYSMVLEIFGLGVDSTAATAVWALSFLVGEQEIQEKLYREINNRTGGQRPVKVVDLKELPYLQAVMKETLRMKPIAPLAVPHVAAKDTTFKGRRIVKGTKVMVNLYAIHHDPNVFPAPYKFMPERFLKDVNSDGRFGDINTMESSLIPFGAGMRICGGVELAKQMVAFALASMVNEFKWDCVSEGKLPDLSEAISFILYMKNPLEAKITPRTKPFRQLEHHHHHH

SEQ ID NO: 24 - DNA, 1476 bp, Papaver somniferum
  misc_feature 1..1476: note = N-terminales Austauschen der Transmembrandomaene (29 Aminosaeuren) mit der Sequenz AKKTSS und N-terminales Flanking MG ; C-terminaler His-tag; Codon-optimiert fuer E. coli; Fuer Sequenz-ID Nr. 23 kodierende Sequenz
  source 1..1476: mol_type = genomic DNA
atgggaatggcaaagaaaacatcgtctaagatcagtttctaccagagcgcaaataccaccgaatggccggccggtccgaaaaccctgccgattattggtaatctgcatcagctgggcggcggcgttccgctgcaggttgctctggcaaatctggccaaagtttatggcggtgcctttaccatttggattggtagctgggttccgatgattgttattagtgatattgataacgcccgtgaagttctggttaataagagcgcagattatagcgcacgcgatgtgccggatattctgaaaattattaccgccaatggtaaaaatatcgccgattgcgatagcggcccgttttggcataatctgaaaaaaggcctgcagagctgcattaatccgagtaatgttatgagcctgagtcgcctgcaggaaaaagatatgcagaatctgattaagagcatgcaggaacgcgcaagccagcataatggcattattaagccgctggatcatgcaaaagaagcaagcatgcgtctgctgagtcgtgtgatttttggccatgattttagcaatgaagatctggttattggcgtgaaagatgccctggatgaaatggttcgtattagcggcctggcaagcctggcagatgcattcaaaattgccaaatatctgccgagtcagcgcaaaaatattcgtgatatgtatgccacccgtgatcgtgtgtataatctgattcagccgcatattgttccgaatctgccggaaaatagttttctgcattttctgaccagccaggattatagtgatgaaattatctatagcatggtgctggaaatttttggtctgggtgttgatagtaccgcagcaaccgcagtgtgggccctgagttttctggtgggcgaacaggaaattcaggaaaaactgtatcgcgaaattaataatcgcaccggtggtcagcgtccggttaaagttgtggatctgaaagaactgccgtatctgcaggcagtgatgaaagaaaccctgcgcatgaaaccgattgcaccgctggccgttccgcatgtggccgcaaaagataccacctttaaaggtcgccgcattgttaaaggcaccaaagtgatggttaatctgtatgccattcatcatgatccgaatgtttttccggcaccgtataaattcatgccggaacgttttctgaaagatgtgaatagcgatggccgctttggcgatattaatacaatggaaagcagcctgattccgtttggtgccggtatgcgtatttgtggcggtgtggaactggcaaaacagatggttgcatttgcactggccagtatggttaatgaattcaaatgggattgtgtgagcgaaggcaaactgccggatctgagtgaagccattagctttattctgtatatgaaaaacccgctggaagccaaaattaccccgcgcaccaaaccgtttcgtcagctcgagcaccaccaccaccaccac

SEQ ID NO: 25 - protein, 494 aa, Papaver somniferum
  REGION 1..494: note = N-terminales Austauschen der Transmembrandomaene (29 Aminosaeuren) mit der Sequenz KKTSSKGR und N-terminales Flanking MG ; C-terminaler His-tag; Codon-optimiert fuer E. coli; Mittels Sequenzierung bestimmte Aminosaeuresequenz
MGMKKTSSKGRKISFYQSANTTEWPAGPKTLPIIGNLHQLGGGVPLQVALANLAKVYGGAFTIWIGSWVPMIVISDIDNAREVLVNKSADYSARDVPDILKIITANGKNIADCDSGPFWHNLKKGLQSCINPSNVMSLSRLQEKDMQNLIKSMQERASQHNGIIKPLDHAKEASMRLLSRVIFGHDFSNEDLVIGVKDALDEMVRISGLASLADAFKIAKYLPSQRKNIRDMYATRDRVYNLIQPHIVPNLPENSFLHFLTSQDYSDEIIYSMVLEIFGLGVDSTAATAVWALSFLVGEQEIQEKLYREINNRTGGQRPVKVVDLKELPYLQAVMKETLRMKPIAPLAVPHVAAKDTTFKGRRIVKGTKVMVNLYAIHHDPNVFPAPYKFMPERFLKDVNSDGRFGDINTMESSLIPFGAGMRICGGVELAKQMVAFALASMVNEFKWDCVSEGKLPDLSEAISFILYMKNPLEAKITPRTKPFRQLEHHHHHH

SEQ ID NO: 26 - DNA, 1482 bp, Papaver somniferum
  misc_feature 1..1482: note = N-terminales Austauschen der Transmembrandomaene (29 Aminosaeuren) mit der Sequenz KKTSSKGR und N-terminales Flanking MG ; C-terminaler His-tag; Codon-optimiert fuer E. coli; Fuer Sequenz-ID Nr. 25 kodierende Sequenz
  source 1..1482: mol_type = genomic DNA
atgggaatgaagaaaacgagcagtaaaggccgtaagatcagtttctaccagagcgcaaataccaccgaatggccggccggtccgaaaaccctgccgattattggtaatctgcatcagctgggcggcggcgttccgctgcaggttgctctggcaaatctggccaaagtttatggcggtgcctttaccatttggattggtagctgggttccgatgattgttattagtgatattgataacgcccgtgaagttctggttaataagagcgcagattatagcgcacgcgatgtgccggatattctgaaaattattaccgccaatggtaaaaatatcgccgattgcgatagcggcccgttttggcataatctgaaaaaaggcctgcagagctgcattaatccgagtaatgttatgagcctgagtcgcctgcaggaaaaagatatgcagaatctgattaagagcatgcaggaacgcgcaagccagcataatggcattattaagccgctggatcatgcaaaagaagcaagcatgcgtctgctgagtcgtgtgatttttggccatgattttagcaatgaagatctggttattggcgtgaaagatgccctggatgaaatggttcgtattagcggcctggcaagcctggcagatgcattcaaaattgccaaatatctgccgagtcagcgcaaaaatattcgtgatatgtatgccacccgtgatcgtgtgtataatctgattcagccgcatattgttccgaatctgccggaaaatagttttctgcattttctgaccagccaggattatagtgatgaaattatctatagcatggtgctggaaatttttggtctgggtgttgatagtaccgcagcaaccgcagtgtgggccctgagttttctggtgggcgaacaggaaattcaggaaaaactgtatcgcgaaattaataatcgcaccggtggtcagcgtccggttaaagttgtggatctgaaagaactgccgtatctgcaggcagtgatgaaagaaaccctgcgcatgaaaccgattgcaccgctggccgttccgcatgtggccgcaaaagataccacctttaaaggtcgccgcattgttaaaggcaccaaagtgatggttaatctgtatgccattcatcatgatccgaatgtttttccggcaccgtataaattcatgccggaacgttttctgaaagatgtgaatagcgatggccgctttggcgatattaatacaatggaaagcagcctgattccgtttggtgccggtatgcgtatttgtggcggtgtggaactggcaaaacagatggttgcatttgcactggccagtatggttaatgaattcaaatgggattgtgtgagcgaaggcaaactgccggatctgagtgaagccattagctttattctgtatatgaaaaacccgctggaagccaaaattaccccgcgcaccaaaccgtttcgtcagctcgagcaccaccaccaccaccac

SEQ ID NO: 27 - protein, 496 aa, Coptis Japonica
  REGION 1..496: note = N-terminales Flanking MG ; C-terminaler His-tag, Codon-optimiert fuer E. coli; Mittels Sequenzierung bestimmte Aminosaeuresequenz
MGMDLQIALFSLIPVILVFILLLKPKYKNLPPGPHPWPLIGNLPILFTNTEVPLHITLANMARTHGPIMILWLGTQPTVMASTAEAAMEILKTHDRIFSARHIRMSFRLKHHIKYSLVWSDCTDYWKLLRKIVRTEIFSPKMLQAQSHVREQKVAELIDFLRSKEGQVVKISQFVFGTLLNILGNVVFSKDVFVYGDETDKGGIQNLIREMLMIGAEPNVAEFYPSLEELDLQGLKKKCDERFIRVMKMWEGTVKERKANRNEESKDMLDVLLANDFNDAQINALFLETFGPGSETSSATIEWVIAELIKSPKEMAKVRKELNEVVGTSTIKESDLPQLPYLQACIKEAMRLHPAAPFLLPRRAAETCEVMGYTIPKNSQVLVNAYAIGRDPKSWKDPSTFWPERFLESDVDFHGAHYQFIPFGSGRRTCVGMPLATRTIPLIVGSLVHNYDFGLPGGNRPEDLKMNEMLSLTLAIDPSLCVVPKARALEHHHHHH

SEQ ID NO: 28 - DNA, 1488 bp, Coptis Japonica
  misc_feature 1..1488: note = N-terminales Flanking MG ; C-terminaler His-tag, Codon-optimiert fuer E. coli; Fuer Sequenz-ID Nr. 27 kodierende Sequenz
  source 1..1488: mol_type = genomic DNA
atgggaatggatctgcagattgccctgttcagcctgattccggttattctggtgttcattctgctgctgaaaccgaaatataaaaatctgccgccgggcccgcatccgtggcctctgattggtaatctgccgattctgttcaccaataccgaagtgccgctgcatattaccctggcaaatatggcccgtacccacggtccgattatgattctgtggctgggtacccagccgaccgttatggccagtaccgcagaagccgcaatggaaattctgaaaacacatgatcgtatcttcagtgcacgccatattcgtatgagcttccgcctgaaacatcatattaaatatagtctggtgtggagcgattgcaccgattattggaaactgctgcgtaaaattgtgcgcaccgaaatcttcagcccgaaaatgctgcaggcccagagtcatgtgcgcgaacagaaagtggcagaactgattgacttcctgcgtagcaaagaaggtcaggtggttaaaattagtcagttcgtgttcggtaccctgctgaatattctgggtaatgttgtgttcagcaaagatgtgttcgtgtatggtgatgaaaccgataaaggtggtattcagaatctgattcgcgaaatgctgatgattggtgcagaaccgaatgttgcagaattctatccgagcctggaagaactggatctgcagggtctgaaaaaaaaatgtgatgaacgcttcattcgcgttatgaaaatgtgggaaggcaccgtgaaagaacgcaaagcaaatcgcaatgaagaaagtaaagatatgctggatgtgctgctggccaatgacttcaatgatgcacagattaatgccctgttcctggaaaccttcggtccgggtagcgaaaccagcagcgcaaccattgaatgggttattgcagaactgatcaaaagcccgaaagaaatggcaaaagtgcgtaaagaactgaatgaagttgttggcaccagtaccattaaagaaagtgatctgccgcagctgccgtatctgcaggcatgtattaaagaagccatgcgcctgcatccggcagccccgttcctgctgccgcgtagagccgcagaaacctgcgaagtgatgggttataccattccgaaaaatagtcaggtgctggttaatgcatatgcaattggtcgtgatccgaaaagctggaaagatccgagtaccttctggccggaacgcttcctggaaagcgatgttgacttccacggtgcacattatcagttcattccgttcggcagcggtcgccgtacctgcgtgggcatgccgctggcaacccgtaccattccgctgattgtgggtagcctggtgcataattatgacttcggcctgccgggtggtaatcgcccggaagatctgaaaatgaatgaaatgctgagcctgaccctggcaattgatccgagtctgtgtgtggttccgaaagcacgtgccctcgagcaccaccaccaccaccac

SEQ ID NO: 29 - protein, 476 aa, Coptis Japonica
  REGION 1..476: note = N-terminales Entfernen der Transmembrandomaene (21 Aminosaeuren) und N-terminales Flanking MG ; C-terminaler His-tag; Codon-optimiert fuer E. coli; Mittels Sequenzierung bestimmte Aminosaeuresequenz
MGMKPKYKNLPPGPHPWPLIGNLPILFTNTEVPLHITLANMARTHGPIMILWLGTQPTVMASTAEAAMEILKTHDRIFSARHIRMSFRLKHHIKYSLVWSDCTDYWKLLRKIVRTEIFSPKMLQAQSHVREQKVAELIDFLRSKEGQVVKISQFVFGTLLNILGNVVFSKDVFVYGDETDKGGIQNLIREMLMIGAEPNVAEFYPSLEELDLQGLKKKCDERFIRVMKMWEGTVKERKANRNEESKDMLDVLLANDFNDAQINALFLETFGPGSETSSATIEWVIAELIKSPKEMAKVRKELNEVVGTSTIKESDLPQLPYLQACIKEAMRLHPAAPFLLPRRAAETCEVMGYTIPKNSQVLVNAYAIGRDPKSWKDPSTFWPERFLESDVDFHGAHYQFIPFGSGRRTCVGMPLATRTIPLIVGSLVHNYDFGLPGGNRPEDLKMNEMLSLTLAIDPSLCVVPKARALEHHHHHH

SEQ ID NO: 30 - DNA, 1428 bp, Coptis Japonica
  source 1..1428: mol_type = genomic DNA
  misc_feature 1..1428: note = N-terminales Entfernen der Transmembrandomaene (21 Aminosaeuren) und N-terminales Flanking MG; C-terminaler His-tag; Codon-optimiert fuer E. coli; Fuer Sequenz-ID Nr. 29 kodierende Sequenz
atgggaatgaaaccgaaatataaaaatctgccgccgggcccgcatccgtggcctctgattggtaatctgccgattctgttcaccaataccgaagtgccgctgcatattaccctggcaaatatggcccgtacccacggtccgattatgattctgtggctgggtacccagccgaccgttatggccagtaccgcagaagccgcaatggaaattctgaaaacacatgatcgtatcttcagtgcacgccatattcgtatgagcttccgcctgaaacatcatattaaatatagtctggtgtggagcgattgcaccgattattggaaactgctgcgtaaaattgtgcgcaccgaaatcttcagcccgaaaatgctgcaggcccagagtcatgtgcgcgaacagaaagtggcagaactgattgacttcctgcgtagcaaagaaggtcaggtggttaaaattagtcagttcgtgttcggtaccctgctgaatattctgggtaatgttgtgttcagcaaagatgtgttcgtgtatggtgatgaaaccgataaaggtggtattcagaatctgattcgcgaaatgctgatgattggtgcagaaccgaatgttgcagaattctatccgagcctggaagaactggatctgcagggtctgaaaaaaaaatgtgatgaacgcttcattcgcgttatgaaaatgtgggaaggcaccgtgaaagaacgcaaagcaaatcgcaatgaagaaagtaaagatatgctggatgtgctgctggccaatgacttcaatgatgcacagattaatgccctgttcctggaaaccttcggtccgggtagcgaaaccagcagcgcaaccattgaatgggttattgcagaactgatcaaaagcccgaaagaaatggcaaaagtgcgtaaagaactgaatgaagttgttggcaccagtaccattaaagaaagtgatctgccgcagctgccgtatctgcaggcatgtattaaagaagccatgcgcctgcatccggcagccccgttcctgctgccgcgtagagccgcagaaacctgcgaagtgatgggttataccattccgaaaaatagtcaggtgctggttaatgcatatgcaattggtcgtgatccgaaaagctggaaagatccgagtaccttctggccggaacgcttcctggaaagcgatgttgacttccacggtgcacattatcagttcattccgttcggcagcggtcgccgtacctgcgtgggcatgccgctggcaacccgtaccattccgctgattgtgggtagcctggtgcataattatgacttcggcctgccgggtggtaatcgcccggaagatctgaaaatgaatgaaatgctgagcctgaccctggcaattgatccgagtctgtgtgtggttccgaaagcacgtgccctcgagcaccaccaccaccaccac

(19) United States
(12) Patent Application Publication
(10) Pub. No.: US 2026/0265799 A1
(43) Pub. Date: Sep. 10, 2026

(54) METHOD FOR PRODUCING CORYTUBERINE AND SALUTARIDINE

(71) Applicant: Universität Graz, Graz (AT)

(72) Inventors: Wolfgang KROUTIL, Graz (AT); Jakob PLETZ, Graz (AT); Emmanuel CIGAN, Graz (AT)

(21) Appl. No.: 19/165,844

(22) PCT Filed: Mar. 6, 2024
(86) PCT No.: PCT/EP2024/055918

§ 371 Date: Sep. 16, 2025

(30) Foreign Application Priority Data
Mar. 17, 2023 (EP) 23162704.3

(51) Int. Cl. C12P 17/12

(52) U.S. Cl. CPC C12P 17/12

(57) ABSTRACT

A method for preparing corytuberine and salutaridine via reticuline as an intermediate, involves the following reaction steps:
a) amidation of 2-(4-hydroxy-3-methoxyphenyl)-N-methylethanamine (12) with 3-hydroxy-4-methoxyphenylacetic acid to yield N-[2-(4-hydroxy-3-methoxyphenyl)-ethyl-N-methyl-3-hydroxy-4-methoxyphenylacetic acid amide (13):

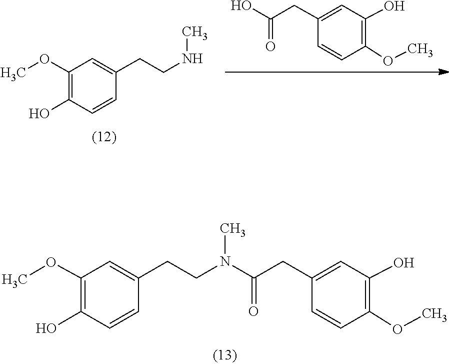

b) Bischler-Napieralski cyclization of the tertiary amide (13) to yield 1,2-dehydro-reticuline (14):

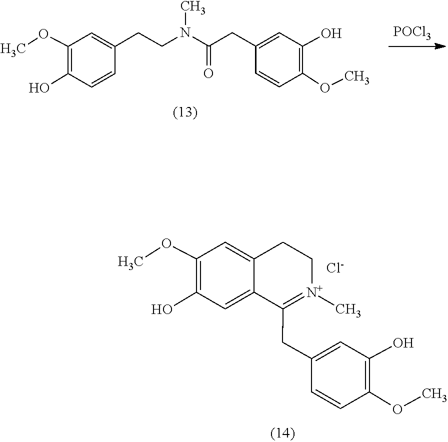

c) biocatalytic enantioselective hydrogenation of 1,2-dehydroreticuline (14) to yield reticuline (15):
c1) using an imine reductase to yield (S)-reticuline (S)-(15) or
c2) using a dehydroreticuline reductase to yield (R)-reticuline (R)-(15):

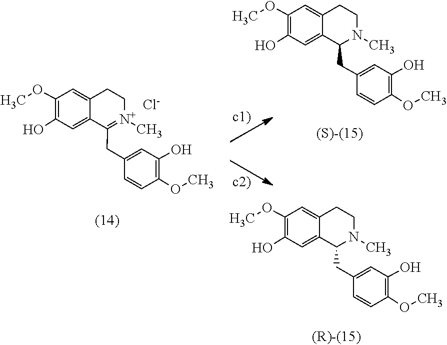

d) biocatalytic oxidative phenol coupling between the two aromatic rings of the respective reticuline:
d1) ortho-ortho coupling of (S′)-reticuline in the presence of corytuberine synthase (CorSyn) to yield corytuberine (16) or
d2) ortho-para coupling of (R)-reticuline in the presence of salutaridine synthase (SalSyn) to yield salutaridine (17):

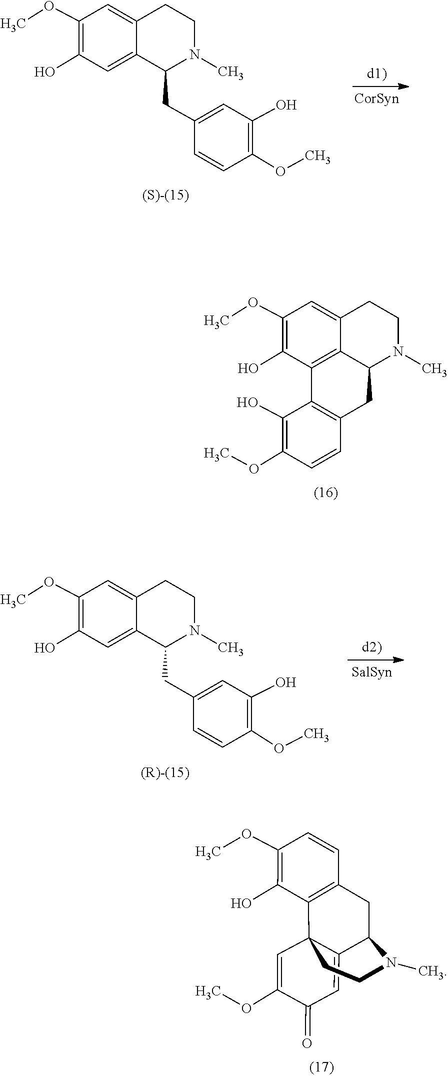

The present invention relates to a method for preparing corytuberine and salutaridine via reticuline as an intermediate.

### PRIOR ART

Alkaloids are nitrogen-containing, mostly biologically active, secondary metabolites. A subclass thereof are benzylisoquinoline alkaloids (BIA), the structures of which serve as precursors to numerous more complex compounds, albeit the molecules based on BIA structures exhibit biological activity as well. Alkaloids are present in almost all classes of organisms where they may play a wide variety of roles, for example, as defense against predators in the plant kingdom. However, these alkaloid structures also play an important role as APIs for the pharmaceutical and medical industries.

One of the most prominent examples of alkaloids is morphine, which humans have been using in various mixtures for more than five thousand years due to the astonishing effectiveness of the opium latex. However, it was not until the beginning of the 19th century that morphine was identified as the main active ingredient in opium extract, rendering it an effective and powerful analgesic. And although the structure of morphine was already elucidated in 1925, to date it has only been available in large quantities through isolation from plant material, so that, despite numerous attempts to entirely synthesize it by organic-chemical or biochemical/biotechnological (mostly based on metabolic engineering) methods, there is still no efficient industrial-scale synthesis process for morphine or its natural precursors, the mono- and dimethyl ether derivatives codeine and thebaine.

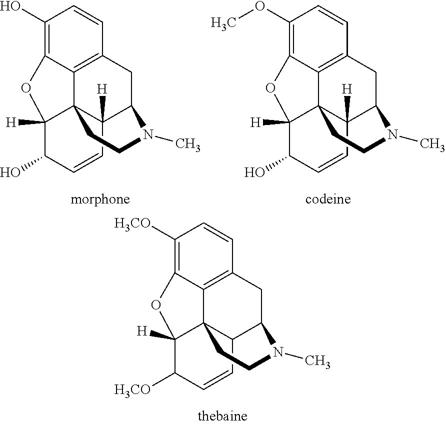

The first practically relevant approach to the synthesis of these morphinan opiates dates back to 1980 by Rice and can be regarded as the first milestone in the field of total synthesis (K. C. Rice, “Synthetic opium alkaloids and derivatives. A short total synthesis of (±)-dihydrothebainone, (±)-dihydrocodeinone, and (±)-nor-dihydrocodeinone as an approach to a practical synthesis of morphine, codeine, and congeners”, J. Org. Chem. 45(15), 3135-3137 (1980), and WO 82/00144 A1). This approach includes, among other things, the 14-step synthesis of the morphine derivative dihydrocodeinone in racemic form with an overall yield of 29% at least, whereafter, however, five further reaction steps are required to obtain the final morphine structure.

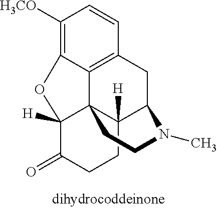

Since then, numerous total syntheses for the preparation of morphinan compounds have been published. The next notable success was achieved by P. Magnus and his co-workers in 2009, who prepared racemic codeine, representing a direct morphine derivative, by means of 13 synthetic steps and with a 20% overall yield (P. Magnus et al., “Concise Syntheses of (−)-Galanthamine and (±)-Codeine via Intramolecular Alkylation of a Phenol Derivative”, J. Am. Chem. Soc. 131(44), 16045-16047 (2009)).

The first relevant stereoselective synthesis route for an opiate was demonstrated by Geffe and Opatz in 2014 (M. Geffe and T. Opatz, “Enantioselective Synthesis of (−)Dihydrocodeine and Formal Synthesis of (−)-Thebaine, (−)-Codeine, and (−)-Morphine from a Deprotonated α-Aminonitrile”, Org. Lett. 16(20), 5282-5285 (2014)). They succeeded in synthesizing (−)-dihydrocodeine by means of 13 synthetic steps and with an overall yield of 20%, starting from the simple starting material gallic acid methyl ester. From this, (−)-codeine and (−)-morphine could be produced in further reaction steps.

Only recently, researchers from research groups led by Qin and Zhong demonstrated a synthetic route that enabled the preparation of (−)-codeine, (−)-naloxone, and (−)oxycodone by means of 12-15 synthesis steps and with 16-34% overall yield, although this was based on non-commercially available starting materials (H. He et al., “Concise total synthesis of opioids”, Org. Chem. Front. 9(9), 2322-2327 (2022)).

The last important publication originates from the research group led by M. G. Banwell and P. Lan, who published a 7-step synthesis for the production of (−)-codeine (L. V. White et al., “Expeditious Access to Morphinans by Chemical Synthesis”, Angew. Chem. Int. Ed. 61(27), e202203186 (2022)). To date, this represents the shortest synthetic route in the field of total synthesis of opiate compounds.

In general, all of the total syntheses mentioned above involve a high number of synthesis steps (except for the publication by L. V. White et al.) and the use of numerous protecting groups. Furthermore, almost all of the syntheses mentioned above involve multiple steps catalyzed by transition metal complexes.

In recent years, biochemical and biotechnological processes for the production of various morphinan alkaloids have been increasingly investigated. For example, the research group led by Christina D. Smolke (Galanie et al., “Complete biosynthesis of opioids in yeast”, Science 349(6252), 1095-100 (2015)) discloses a complex pathway design, also known as metabolic engineering, by which thebaine and dihydrocodeinone can be produced using 21 or 23 enzymes, respectively, starting with a sugar fermentation using baker's yeast (Saccharamyces cerevisiae). Although this work can certainly be considered a milestone in biological synthesis, it represents no more than a proof of concept, since the yields were only between 0.3 and 6.4 μg/l and thus several orders of magnitude below economically practicable values.

One year later, the research group led by Hiromichi Minami (Nakagawa et al., “Total biosynthesis of opiates by stepwise fermentation using engineered Escherichia coli”, Nature Communications 7(1), 10390 (2016)) succeeded in increasing the yields of thebaine and dihydrocodeinone by three orders of magnitude (to 2.1 and 0.36 mg/I, respectively) by performing the biosynthesis with Escherichia coli instead of baker's yeast, in a stepwise culturing process using four separate E. coli cultures, from which the respective product was extracted and used as a substrate for the next fermentation step.

In the latter studies, highly complex enzymes, namely cytochromes P450 (CYP) from higher plants, were used in eukaryotic as well as prokaryotic cells, with the latter generating even higher yields.

In addition, there are several studies dealing with the production of an essential intermediate of the natural synthesis of these alkaloids, namely the benzylisoquinoline alkaloid (S)-reticuline:

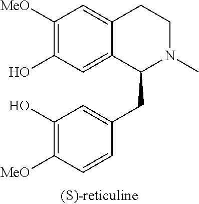 [SMILES: COc1ccc(C[C@H]2c3cc(O)c(OC)cc3CCN2C)cc1O]

While the majority of these, again, only achieved yields in the range of a few micrograms to milligrams per liter, two years ago, a research group (Pyne et al., “A yeast platform for high-level synthesis of tetrahydroisoquinoline alkaloids”, Nat. Commun. [Online]2020, 3337) succeeded in synthesizing this compound using Saccharomyces cerevisae cultures with a yield of 4.6 g/I, thus revealing a synthetically relevant biotechnological approach for the first time.

In nature, (S)-reticuline is oxidized in the capsules of the opium poppy (Papaver somniferum) in a two-step enzymatic epimerization first to 1,2-dehydroreticuline, which is then reduced to (R)-reticuline (which is subsequently converted to morphine or its derivatives such as codeine and thebaine):

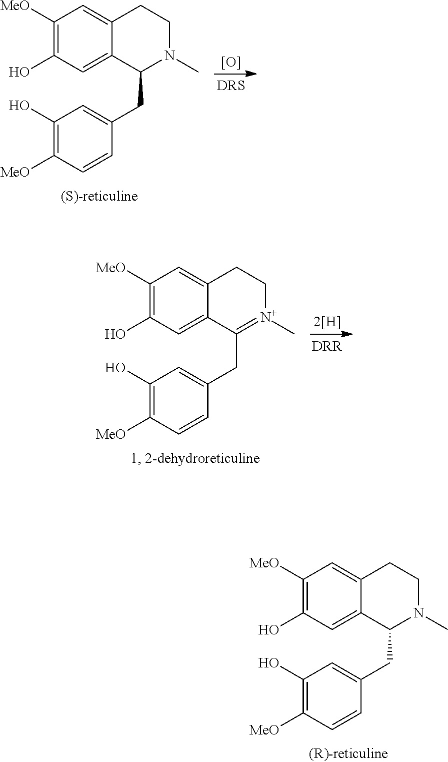

As recently discovered by the research group led by Peter J. Facchini (Farrow et al., “Stereochemical inversion of (S)-reticuline by a cytochrome P450 fusion in opium poppy”, Nat. Chem. Biol. 11(9), 728-32 (2015)), both reactions are catalyzed by the same fusion protein, (S)-reticuline epimerase, which is also known as STORR (“(S)- to (R)-reticuline”) protein. This consists of two domains: an N-terminal domain, which includes the cytochrome P450 enzyme dehydroreticuline synthase (DRS), and a Cterminal domain, which represents the aldo-keto reductase dehydroreticuline reductase (DRR).

Furthermore, a few publications exist on the production of morphine opiates such as morphine, codeine, and thebaine using a combination of organic chemical synthesis and enzymatic catalysis, with the latter consistently being used first to produce various starting components for the subsequent chemical syntheses of the desired compounds. For example, the research group led by Tohru Fukuyama (Koizumi et al., “Total Synthesis of (−)-Morphine”, Chem. Asian J. 5(10), 2192-2198 (2010)) discloses a 17-step synthesis for the production of morphine with a total yield of 5%, with only an early precursor being produced by enzymatic catalysis using a lipase. And Bedard and Hudlicky describe in “Chapter 2—Enzymatic dihydroxylation of aromatic compounds: Nature's unique reaction and its impact on the synthesis of natural products”, Strategies and Tactics in Organic Synthesis, Harmata, M. (ed.), Academic Press, Vol. 15, pp. 53-97 (2021), the enzymatic dihydroxylation of phenethyl bromide to produce a starting compound for the subsequent chemical synthesis of codeine.

Several organic chemical synthesis routes have also been published for the preparation of various benzylisoquinoline alkaloids as precursors in the synthesis of morphinan structures, which typically involve a Pictet-Spengler reaction for preparing heterocycles by reacting aromatic-substituted ethylamines with aldehydes, followed by dehydrative cyclization. This typically requires chiral catalysts and numerous synthetic steps, and both overall yields and stereoselectivities are typically low (see Calcaterra et al., “The Pictet-Spengler Reaction Updates Its Habits”, Molecules 25(2), 414 (2020)).

In nature, this reaction is catalyzed by the Pictet-Spenglerase norcoclaurine synthase (NCS), whereby the benzylisoquinoline alkaloid (S)-norcoclaurine is available efficiently and in optical purity:

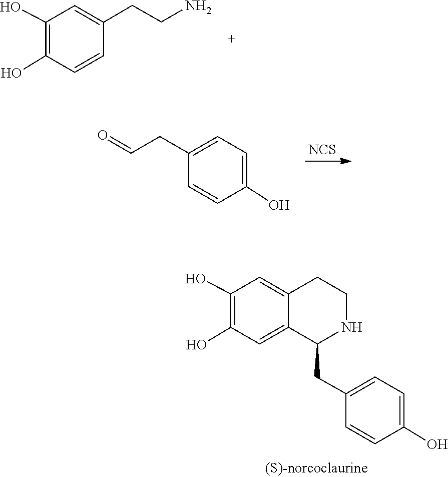 [SMILES: NCCc1ccc(O)c(O)c1 ; O=CCc1ccc(O)cc1 ; "NCS" ; Oc1ccc(C[C@@H]2NCCc3cc(O)c(O)cc32)cc1]

Only recently, the research group of the present inventors led by Wolfgang Kroutil succeeded in carrying out this NCS-catalyzed biotransformation using in vitro biocatalysis (see Cigan et al., “The role of biocatalysis in the asymmetric synthesis of alkaloids—an update”, RSC Advances 11(45), 28223-28270 (2021)). However, they had to detect that NCS requires a phenolic OH group in the meta position to the ethylamine, making this reaction unsuitable for the production of methoxy derivatives such as reticuline.

Against this background, the object of the invention was to develop a new process by which benzylisoquinoline alkaloids and other essential intermediates in the synthesis of various alkaloids can be obtained in a less complex manner and in good yields by means of a combination of enzymatic catalysis and organic-chemical synthesis.

### SUMMARY OF THE INVENTION

The present invention achieves this object by providing a method for preparing corytuberine and salutaridine via the benzylisoquinoline alkaloid reticuline as an intermediate, the process comprising the following reaction steps:
a) amidation of 2-(4-hydroxy-3-methoxyphenyl)-N-methylethanamine (12) with 3-hydroxy-4-methoxyphenylacetic acid to yield N-[2-(4-hydroxy-3-methoxyphenyl)ethyl-N-methyl-3-hydroxy-4-methoxyphenylacetic acid amide (13):

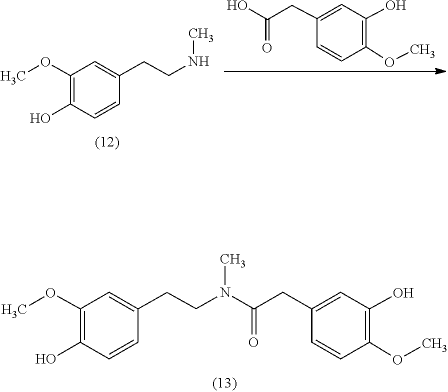

b) Bischler-Napieralski cyclization of the tertiary amide (13) to yield 1,2-dehydroreticuline (14):

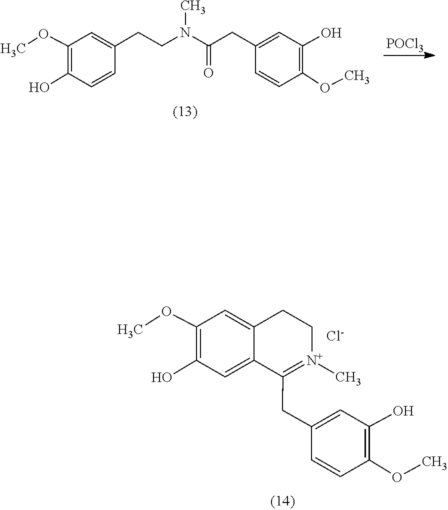

c) biocatalytic enantioselective hydrogenation of 1,2-dehydroreticuline (14) to yield reticuline (15), either
c1) using an imine reductase to yield (S)-reticuline (S)-(15) or
c2) using a dehydroreticuline reductase to yield (R)-reticuline (R)-(15):

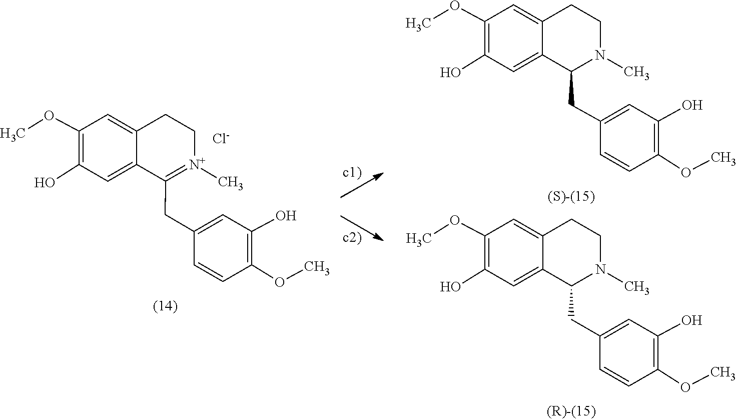

d) biocatalytic oxidative phenol coupling between the two aromatic rings of the respective reticuline, namely either
d1) ortho-ortho coupling of (S)-reticuline in the presence of corytuberine synthase (CorSyn) to yield corytuberine (16) or
d2) ortho-para coupling of (R)-reticuline in the presence of salutaridine synthase (SalSyn) to yield salutaridine (17):

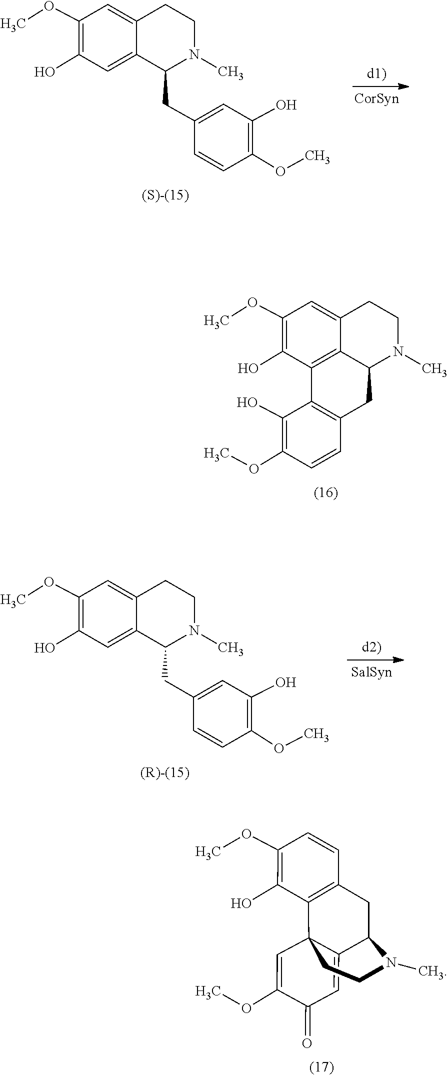

In this way, according to the present invention, it is possible, using a few synthesis steps, to first produce the benzylisoquinoline alkaloid reticuline—selectively in the (S) or (R) configuration—and from this, in just one step, either corytuberine or salutaridine, the first of which is already an aporphine alkaloid and the second of which is a precursor for the synthesis of morphinan alkaloids. In this way, for example, the aporphine alkaloid bulbocapnine can be formed from corytuberine (16) by cyclization:

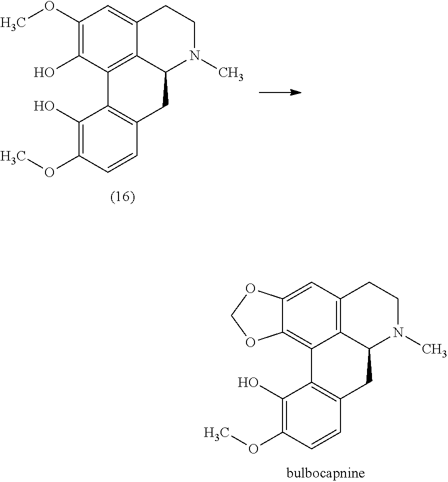

And salutaridine can first be reduced (e.g. using an oxidoreductase) to yield salutaridinol, which can then be acetylated (e.g. using an O-acetyltransferase and acetyl-CoA) and thereafter be cyclized, with the acetate being cleaved, to yield thebaine:

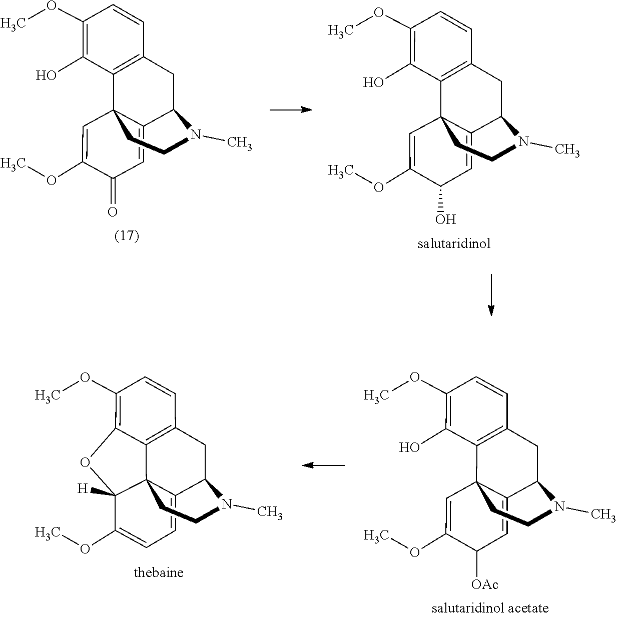

Furthermore, the initial organic chemical reactions can already be carried out with high yields, and the two subsequent biocatalyses each produce the desired products in practically quantitative yield and with almost 100% enantiomeric purity (>99% ee).

In addition, the starting product 2-(4-hydroxy-3-methoxyphenyl)-N-methyl-ethanamine (12) is accessible by a direct methylation step from commercially available 2-(4-hydroxy-3-methoxyphenyl)ethanamine, although, in preferred embodiments of the invention, it is instead prepared using three simple synthesis steps from a considerably less expensive starting product, as will be explained in more detail later.

The amidation in step a) could also be carried out in a generally known manner using the acid chloride of 3-hydroxy-4-methoxyphenylacetic acid, although this would require an additional step for its synthesis from the commercially available free acid. Therefore, according to the present invention, the amide (13) is prepared as indicated above from the free acid and the secondary amine (12) by means of a condensation reaction. This can be achieved, for example, by intensely heating the reactants, but is preferably carried out using a carbodiimide to activate the acid, with 1-ethyl-3-(3-dimethylaminopropyl)carbodiimide (EDC) being particularly preferably used as the carbodiimide, since the urea formed as a by-product can easily be separated. In particular, hydroxyiminocyanoacetic acid ethyl ester (Oxyma) is added as an additive, which prevents the formation of undesirable by-products, e.g. the unreactive N-acylurea, thus increasing the yields of the desired amide.

The Bischler-Napieralski cyclization in step b) is carried out in an aprotic polar solvent, preferably in acetonitrile, more preferably under reflux, and provides 1,2-dehydroreticuline (14) in high yield.

Its biocatalytic enantioselective hydrogenation in step c1) is carried out using an imine reductase, for which a variety of enzymes are available. In preferred embodiments of the invention, one of the 88 imine reductases described by Zhu et al. in “Enantioselective Synthesis of 1-Aryl-Substituted Tetrahydroisoquinolines Employing Imine Reductase”, ACS Catalysis 7(10), 7003-7007 (2017), for the reduction of some 1-aryldihydroisoquinolines is used. However, even more preferably, according to the present invention, the imine reductase IR45 F190M-W191F disclosed by the same research group in Yang et al., “Biosynthesis of plant tetrahydroisoquinoline alkaloids through an imine reductase route”, Chemical Science 11(2), 364-371 (2020), is used, since it has proven effective in the reduction of various 1-phenyldihydroisoquinolines, but also of several 1-benzyldihydroisoquinolines, which have a similar structure to 1,2-dehydroreticuline.

Therefor, a recombinant enzyme heterologously expressed by cells transformed with the corresponding gene is preferably used, and even more preferably, a recombinant enzyme heterologously expressed by E. coli cells, in particular competent BL21(DE3) cells, is used in the form of the lyophilized cells or a cell-free extract thereof. The best results were achieved using a cell-free extract of transformed BL21(DE3) cells cultured in terrific broth (TB) medium.

The other conditions of the hydrogenation in step c1) are not specifically restricted, but preferably one or more or all of the following conditions i) to v) are selected:
i) 0.1 M KPi buffer is used as a solvent;
ii) the dehydroreticuline concentration is 5-30 mM, more preferably 10-15 mM, in particular 10 mM;
iii) the pH is 7-8.5, more preferably 7.5-8.0;
iv) NADPH/NADP⁺ is used as a cofactor, in particular at a concentration of 1 mM;
v) glucose dehydrogenase is used as a coenzyme, in particular at a concentration of 2 mg/mL, and D-glucose is used as a cosubstrate, in particular at a concentration of 9 mg/mL.

The biocatalytic enantioselective hydrogenation of 1,2-dehydroreticuline to yield (R)-reticuline in step c2) under dehydroreticuline reductase catalysis is preferably carried out using the dehydroreticuline reductase from Papaver somniferum (PsDRR), which is part of the (S)-reticuline epimerase in opium poppy capsules and also catalyzes this hydrogenation reaction in nature. In this case, too, a recombinant enzyme heterologously expressed by cells transformed with the corresponding gene is particularly preferred, even more preferably an enzyme expressed by E. coli cells, particularly an enzyme expressed by competent Arctic Express (DE3) cells, is used in the form of the lyophilized cells or a cell-free extract thereof. The best results were achieved using transformed Arctic Express (DE3) cells cultured in lysogeny broth (LB) medium, more precisely using the lyophilized whole cells.

The other conditions of the hydration in step c2) are not specifically restricted, but preferably one or more or all of the following conditions i) to vi) are selected:
i) 0.1 M Tris-HCl buffer or glycine-NaOH buffer is used as a solvent;
ii) 5-10 vol %, in particular 10 vol %, DMSO are used as a cosolvent;
iii) the dehydroreticuline concentration is 10-30 mM, in particular 10-20 mM; iv) the pH is 8-8.5 (Tris-HCl) or 9-9.5 (glycine-NaOH);
v) NADPH/NADP⁺ is used as a cofactor, in particular at a concentration of 1 mM;
vi) glucose dehydrogenase is used as a coenzyme, in particular at a concentration of 2 mg/mL, and D-glucose is used as a cosubstrate, in particular at a concentration of 9 mg/mL.

In further preferred embodiments of the present invention, the biocatalytic oxidative phenol coupling between the two aromatic rings of (S)-reticuline in step d1) is carried out using a corytuberine synthase, preferably the corytuberine synthase from Coptis japonica (CjCorSyn) or a modified version thereof, together with the cytochrome P450 reductase 2 from Arabidopsis thaliana (AtR2) as a redox partner in order to optimize the efficiency of the catalytic system. In particularly preferred embodiments, the corytuberine synthase and AtR2 are used as recombinant enzymes heterologously expressed by cells transformed with the corresponding genes, even more preferably as enzymes expressed by E. coli cells, particularly preferred, again, expressed by competent BL21(DE3) cells, in the form of whole cells or a cell-free extract thereof. The best results were achieved using transformed BL21(DE3) cells cultured in TB medium, or more precisely, the resuspended whole cells.

The other conditions of the ortho-ortho phenol coupling in step d1) are not specifically restricted, but preferably one or more or all of the following conditions i) to vii) are selected:
i) 0.1 M KPi buffer is used as a solvent;
ii) the enzymes are used in the form of resuspended whole cells;
iii) 0.1-5 vol %, in particular 0.2-1 vol %, DMSO are used as a cosolvent;
iv) the reticuline concentration is 0.1-5 mM;
v) the pH is 7.5-8.0;
vi) NADPH/NADP⁺ is used as a cofactor, in particular at a concentration of 0.05 mM;
vii) a glucose dehydrogenase preparation is used as a coenzyme, in particular at a concentration of 2 mg/mL, and D-glucose is used as a cosubstrate, in particular at a concentration of 50 mM.

According to a further preferred embodiment, the biocatalytic oxidative phenol coupling between the two aromatic rings of (R)-reticuline in step d2) is carried out using the salutaridine synthase from Papaver somniferum (PsSalSyn), a chimeric version thereof which comprises at the N-terminal end a peptide sequence of another cytochrome P450 enzyme, namely cheilanthifoline synthase CYP719A5 (CFS_PsSalSyn), or a construct in which the N-terminal membrane part has been exchanged for a hydrophilic peptide sequence (Nswap_KK_SalSyn). Even more preferably, salutaridine synthase is used together with the cytochrome P450 reductase 2 from Arabidopsis thaliana (AtR2) as a redox partner. In particularly preferred embodiments, the salutaridine synthase and AtR2 are used as recombinant enzymes heterologously expressed by cells transformed with the corresponding genes, even more preferably as enzymes expressed by E. coli cells, particularly preferred, again, expressed by competent BL21(DE3) cells, in the form of lyophilized cells or a cell-free extract thereof. The best results were achieved using transformed BL21(DE3) cells cultured in TB medium, or more precisely, the resuspended whole cells.

The other conditions of the ortho-para phenol coupling in step d2) are not specifically restricted, but preferably one or more or all of the following conditions i) to vii) are selected:
i) 0.1 M KPi buffer is used as a solvent;
ii) the enzymes are used in the form of resuspended whole cells;
iii) 0.1-5 vol %, in particular 0.2-2 vol %, DMSO are used as a cosolvent;
iv) the reticuline concentration is 0.1-0.5 mM;
v) the pH is 7.5-8.0;
vi) NADPH/NADP⁺ is used as a cofactor, in particular at a concentration of 0.05 mM;
vii) a glucose dehydrogenase preparation is used as a coenzyme, in particular at a concentration of 2 mg/mL, and D-glucose is used as a cosubstrate, in particular at a concentration of 50 mM.

As previously mentioned, in particularly preferred embodiments of the method of the present invention, the starting product 2-(4-hydroxy-3-methoxyphenyl)-N-methyl-ethanamine (12) used in step a) is previously prepared starting from 4-allyl-2-methoxyphenol (eugenol) by the following reaction steps:
I) ozonolysis of 4-allyl-2-methoxyphenol (9) to yield 4-hydroxy-3-methoxyphenylacetaldehyde

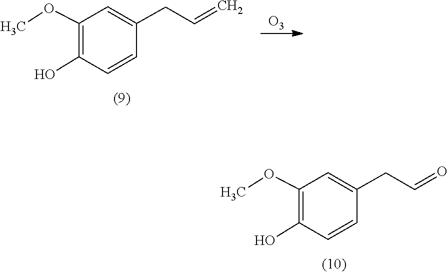

II) reductive amination of the aldehyde (10) with N-benzylmethanamine to yield the benzyl-protected intermediate N-benzyl-2-(4-hydroxy-3-methoxyphenyl)-N-methyl-ethanamine (11):

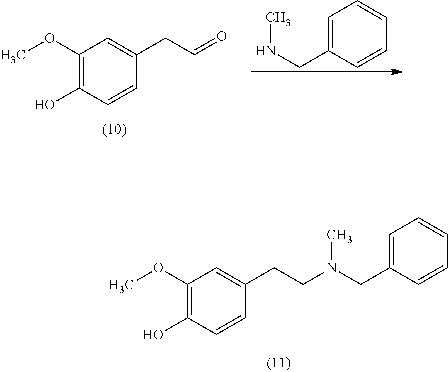

III) hydrogenolysis to remove the benzyl-protecting group to obtain the desired amine (12):

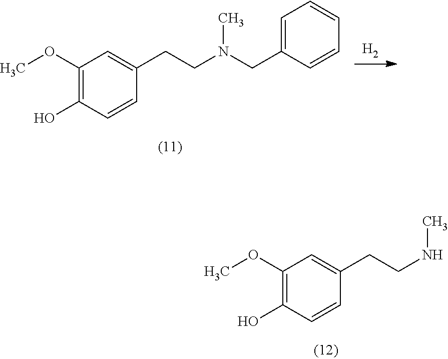

Eugenol (9), used here as starting material, is commercially available at a considerably lower price, and the products of reaction steps I) to III) can all be prepared in very good yields—especially after optimizing the reactions in the following manner.

Preferably, the ozonolysis of eugenol (9) in step I) is carried out in an organic solution by introducing a mixture of O₃/O₂ at a temperature <0° C., more preferably at −15° C., and subsequently quenching the ozonide using a reducing agent, more preferably dimethyl sulfide; and/or
the reductive amination of the aldehyde (10) in step II) is carried out in an organic solution in the presence of a reducing agent, more preferably a borohydride, in particular NaBH(OAc)₃, with ice bath cooling;
and/or
the hydrogenolysis in step III) is carried out in an alcoholic solution in the presence of a palladium catalyst, more preferably Pd/C, and HCl at room temperature; and the other conditions are not specifically restricted and can be optimized separately in each individual case.

The reductive amination of homovanillin (10) with N-benzylmethanamine to yield the benzyl-protected intermediate (11) is preferable because during the direct reaction with methylamine to yield the amine (12), various side reactions can occur: in the organic-chemical synthesis, overalkylation can easily occur, and in the enzymatic catalysis in a basic aqueous solution, the aldehyde (10) tends to spontaneous dimerization.

In a particularly preferred embodiment of the invention, steps I) and II) are carried out as a one-pot reaction, which eliminates the need for isolating homovanillin (10), which increases the overall yield.

### EXAMPLES

The present invention will be described in more detail below with reference to representative, non-limiting examples which are given for illustration purposes only.

### Materials and Methods

All chemicals were purchased from Sigma Aldrich, Acros Organics, Alfa Aesar, or ABCR and used without further purification. All solvents were purchased from Roth. Solvents marked “abs.” were dried by dehydration. Laboratory-grade water was produced using in-house equipment (desalinator). Gene strands were purchased from Thermo Fisher Scientific at Invitrogen, and plasmids containing genes were purchased either from BioCat GmbH or General Biosystems. Unless otherwise stated, the specified equipment was used under standard conditions, and all terms, chemical names, and abbreviations have the usual meaning well-known to average experts.

The reactions were monitored by thin-layer chromatography (TLC), using Merck silica/aluminum plates (Silica Gel 60 F₂₃₄ Aluminum TLC plates, 20×20 cm) as the stationary phase. Detection was performed with UV light (254 nm) or staining with “CAM Blue” (ceric ammonium molybdate) solution, which was obtained by dissolving 2.5 g of ammonium molybdate tetrahydrate and 1 g of ammonium cerium(IV) sulfate dihydrate in 90 mL of distilled water with stirring, followed by the addition of 10 mL of conc. H₂SO₄. All GC-MS measurements were performed using an Agilent 7890A GC system equipped with an Agilent 5975C mass selective detector (electron impact, 70 eV) and an HP-5-MS column (30 m×0.25 mm×0.25 μm film) using He as the carrier gas at a flow rate of 0.55 mL/min. GC-FID analyses were performed using an Agilent 7890A GC system and an HP-5 column (30 m×0.32 mm×0.25 μm film) using H₂ as the carrier gas at a flow rate of 0.55 mL/min. Low-resolution mass spectra were recorded on an Agilent Technologies 6120 Quadrupole LC/MS detector combined with an Agilent Technologies 1260 Infinity HPLC system equipped with a Kinetex 2.6 μm C-18 100A column (50×4.6 mm, 2.6 μm). Water/acetonitrile (+0.1 vol % formic acid) was used as the eluent. HPLC-UV analyses were performed on a Shimadzu HPLC system equipped with a Luna 5 μm C-18 100A column (250×4.6 mm, 5 μm) [DGU-20A (degasser), LC-20A (pump), SIL-20A (autosampler), CTO-20AC (column oven), SPD-M20A (detector), CBM-20AC (controller)] using different eluent systems.

### Example 1

### Preparation of 4-hydroxy-3-methoxyphenylacetaldehyde (Homovanillin) (10) from 4-allyl-2-methoxyphenol (Eugenol) (9)

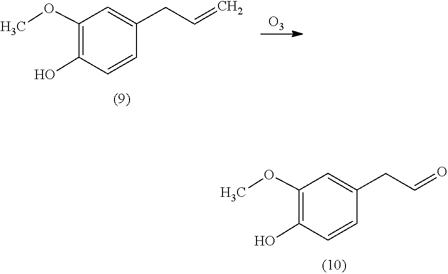

A three-necked round-bottom flask (500 mL) equipped with a magnetic stir bar was charged with eugenol (9) (5.00 g, 30.5 mmol, 1 eq.), followed by CH₂Cl₂ (250 mL). The resulting yellow solution was cooled to −15° C. using a cryostat in an EtOH bath, after which an O₃/O₂ mixture was passed through the solution for 6 h through a sintered glass frit. After complete consumption of the starting material (TLC, EtOAc/cyclohexane=3:1, Rf=0.54), nitrogen was passed through the solution for 10 min to remove excess ozone. The reactive intermediate was quenched by the addition of Me₂S (11.2 mL, 153 mmol, 5 eq.) at −15° C., and after 15 min, the mixture was allowed to warm to room temperature. After complete conversion of the hydroperoxyacetal (TLC, cyclohexane/EtOAc=1:1, Rf=0.36), the mixture was concentrated in vacuo. The product was purified by column chromatography (column: 30×6 cm, cyclohexane/EtOAc=3:1 (v/v). Yield: 3.77 g (22.7 mmol, 74% of theory) of a pale yellow solid.

### Example 2

### Preparation of N-benzyl-2-(4-hydroxy-3-methoxyphenyl)-N-methylethanamine (11) from 3-hydroxy-4-methoxyphenylacetaldehyde (10)

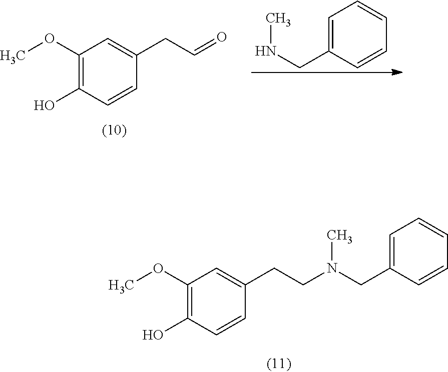

A dry 500 mL round-bottom flask equipped with a magnetic stir bar was charged with 4.00 g (24.1 mmol, 1 eq.) of 2-(4-hydroxy-3-methoxyphenyl)acetaldehyde (10) and 240 mL of CH₂Cl₂. The pale yellow solution was cooled to 0° C. using an ice bath, after which 4.80 mL (36.1 mmol, 1.5 eq.) of N-methyl-1-phenylmethanamine were slowly added. 10.4 g (48.1 mmol, 2 eq.) of NaBH(OAc)₃ were added in portions over a period of 3 min, the flask was sealed with a septum, and the mixture was allowed to stir in the thawing ice bath. After 150 min, incomplete conversion of the aldehyde was detected by TLC, so another portion of NaBH(OAc)₃ (2.60 g, 0.5 eq.) was added at room temperature. Complete conversion of the starting material was determined after a total of 18 h, and the reaction mixture was quenched by adding saturated aqueous NaHCO₃ solution and solid NaHCO₃. The solvents were removed on a rotary evaporator (bath temperature: 50° C.), and the product was purified by two column chromatography steps (1^{st} column: 17×4.8 cm, CH₂Cl₂/acetone=1:1; 2^{nd} column: 16×4.8 cm, CH₂Cl₂/acetone=2:1). Yield: 5.15 g (19.0 mmol, 79% of theory) of a light brown solid.

### Example 3

### Preparation of N-benzyl-2-(4-hydroxy-3-methoxyphenyl)-N-methylethanamine (11) from 4-allyl-2-methoxyphenol (9) in a One-Pot Process

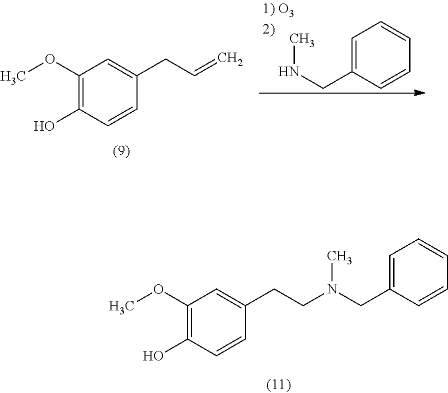

First, homovanillin (10) was prepared as described in Example 1. After complete consumption of the starting material (TLC, EtOAc/cyclohexane=3:1, Rf=0.54), nitrogen was passed through the solution for 10 min to remove excess ozone. The reactive intermediate was quenched by the addition of Me₂S (11.2 mL, 153 mmol, 5 eq.) at −15° C., and after 15 min, the mixture was allowed to warm to room temperature and stirred overnight. The resulting pale yellow solution was cooled to 0° C. using an ice bath, after which the reaction of the intermediate (10) to the title compound (11) and its workup and purification as in Example 2 with 6.10 mL (45.9 mmol, 1.5 eq.) of N-methyl-1-phenylmethanamine and 25.7 g (122.4 mmol, 4 eq.) of NaBH(OAc)₃ were carried out as in Example 2. Yield: 6.6 g (24.5 mmol, 80% of theory) of a light brown solid.

### Example 4

### Preparation of 2-(4-hydroxy-3-methoxyphenyl)-N-methylethanamine (12)

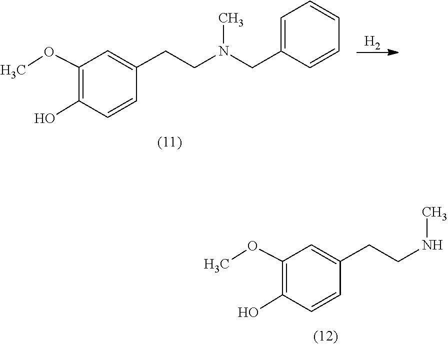

A 1000 mL round-bottom flask equipped with a magnetic stir bar was charged with 4.35 g (16.0 mmol, 1 eq.) of N-benzyl-2-(4-hydroxy-3-methoxyphenyl)-N-methyl-ethanamine (11), 160 mL of 2-propanol, 2.7 mL (32.0 mmol, 2 eq.) of conc. aqueous HCl (12 M), and 218 mg of Pd/C (10%). The mixture was vigorously stirred in hydrogen atmosphere at 22° C. until complete conversion of the starting material was observed after 3 d (TLC). The mixture was filtered through a 6 cm pad of Celite, and the pad was rinsed successively with 2-propanol (500 mL), 2-propanol/H₂O (1:1, 300 mL), and MeOH (400 mL). The solvents were removed on a rotary evaporator, and the product was dried in vacuo to obtain (12) as its hydrochloride. Yield: 3.13 g (14.4 mmol, 90% of theory) of a brown solid.

### Example 5

### Preparation of N-(4-hydroxy-3-methoxyphenethyl)-2-(3-hydroxy-4-methoxyphenyl)-N-methylacetamide

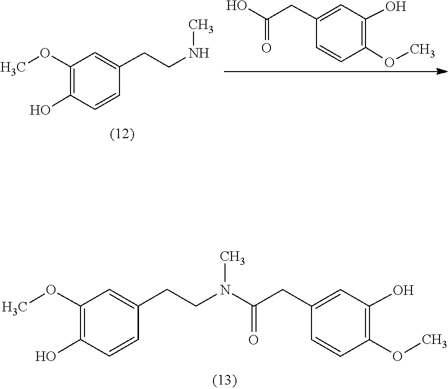

A dry 250 mL round-bottom flask equipped with a magnetic stir bar was charged with 560 mg (2.57 mmol, 1.05 eq.) of the hydrochloride of (12) obtained in Example 4, 20 mL of DMF, and 346 mg (2.44 mmol, 1 eq.) of Oxyma Pure. The solution was cooled to 0° C. using an ice bath, after which 0.34 mL (2.44 mmol, 1 eq.) of Et₃N, followed by 520 mg (2.69 mmol, 1.1 eq.) of 1-ethyl-3-(3-dimethylaminopropyl)carbodiimide hydrochloride (EDC·HCl) were added. To the vigorously stirred solution, 463 mg (2.44 mmol, 1 eq.) of 2-(3-hydroxy-4-methoxyphenyl)acetic acid in 4 mL of DMF were added dropwise over a period of 3 h using a syringe pump. Complete conversion of the starting material was observed after 18.5 h (TLC), and the solvent was removed on a rotary evaporator. The remaining solid was taken up in 70 mL of EtOAc and washed with 1 M aqueous HCl solution (3×50 mL). The combined aqueous washing solution was neutralized with NaHCO₃ and extracted again with EtOAc (3×70 mL). The organic phases were combined with the previous organic phases and concentrated on a rotary evaporator. The product was purified by column chromatography (column: 26×3.3 cm, EtOAc/cyclohexane=2:1, 4:1 (v/v). The product was dried under oil pump vacuum (caution: intense foaming!). Yield: 807 mg (2.34 mmol, 96% of theory) of a colorless, glassy solid.

### Example 6

### Preparation of 1,2-dehydroreticuline (14)

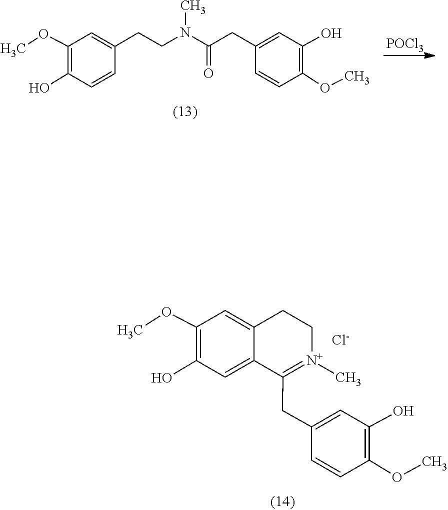

A 250 mL three-necked round-bottom flask equipped with a magnetic stirrer, an air cooler, a pressure equalization, and a gas valve was purged with argon for 45 min. Following this, 1.00 g (2.90 mmol, 1 eq.) of N(4-hydroxy-3-methoxyphenethyl)-2-(3-hydroxy-4-methoxyphenyl)-N-methylacetamide (13) in 30 mL of dry MeCN, followed by 824 μL (8.69 mmol, 3 eq.) of POCl₃ were added in countercurrent Ar. The colorless solution was heated to reflux, and after 4 h, complete conversion of the starting material was observed (TLC). 2.5 g of Celite were added, and the solvent was removed on a rotary evaporator. The product was purified by two column chromatography steps (1^{st} column: 30×1.8 cm, CHCl₃/MeOH/TFA=14:2:1, 12:2:1, 10:2:1 (vol.); 2^{nd} column: 30×1.8 cm, CHCl₃/MeOH/TFA=8:1:1, 7:1:1, 6:1:1, 5:1:1, 4:1:1 (vol.)). The product was dissolved in MeOH, filtered through cotton wool, and the solvent was removed in vacuo. The product was then dissolved in distilled water (50 mL), filtered through a pad of Celite (2 cm), treated with 12 M HCl (500 μL), and dried in vacuo. Yield: 821 mg (2.26 mmol, 78% of theory) of a light yellow powder.

### Reference Example 1

### Preparation of the Mutant Imine Reductase IR45 F190M-W191F (SEQ ID No: 3)

A) Transformation of Competent BL21(DE3) E. coli Cells

Plasmid pET-28a(+), which contains the gene encoding the imine reductase IR45 F190M-W191F from Streptomyces aurantiacus (SEQ ID No: 4), was purchased from BioCat GmbH, and 2 μL thereof were mixed with 100 μL of chemically competent BL21(DE3) E coli host cells in 1.5 mL microcentrifuge tubes. The mixtures were incubated on ice for 30 min. The competent cell/DNA mixtures were then shock-heated at 42° C. for 10 s. 700 μL of autoclaved LB medium were added to each sample, followed by shaking at 37° C., 300 rpm, for 1 h. Subsequently, 100 μL of the mixture were applied to previously prepared kanamycin-containing agar plates, which were placed in a 37° C. incubator overnight.

### B) Preparation of Overnight Cultures

50 mL plastic tubes were filled with 10 mL of LB medium, and 10 μL of kanamycin solution (50 mg/mL, final concentration: 50 μg/mL) were added. A single culture from each agar plate was taken for inoculation and cultured overnight at 30° C. and 120 rpm.

### C) Main Culture

The enzyme was produced in both LB and TB medium.

For each batch, either 330 mL of autoclaved LB medium or 300 mL of autoclaved TB medium supplemented with 30 mL of KH₂PO₄/K₂HPO₄ buffer (10 mL per 100 mL of medium) were each supplemented with 330 μL of kanamycin solution (50 mg/mL, 100 μL per 100 mL of medium) and treated with 3.3 mL of the respective overnight culture (1 mL per 100 mL of medium). Cell growth proceeded at 37° C., 120 rpm, until the OD₆₀₀ value was between 0.6 and 0.8. The cultures were then induced with IPTG (1 M stock solution, final concentration: 0.5 mM). The cell cultures were shaken overnight at 20° C., 120 rpm.

To obtain the enzymes, the cultures were centrifuged (5000 rpm, 4° C., 20 min), resuspended in distilled water, and centrifuged again (5000 rpm, 4° C., 20 min). The pellets were resuspended in 10 mL of water per gram of wet pellet. The resuspensions (LB or TB medium culture) were either sharp-frozen and lyophilized as whole cells or subjected to pulsed sonication for 5 min and centrifuged (14000 rpm, 4° C., 20 min). In the latter case, the pellets were discarded, and the supernatant was sharp-frozen and lyophilized. The resulting lyophilized cell-free extracts (CFE) and lyophilized whole cells were stored at −20° C.

### Example 7

### Preparation of (S)-reticuline (S)-(15)

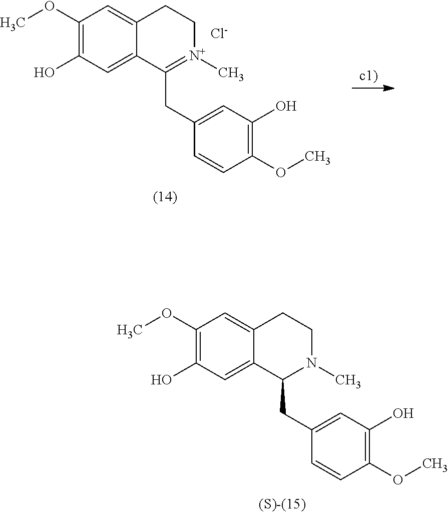

Microcentrifuge tubes (1.5 mL) were charged with 5 mg of the respective IR45 F190M-W191 F enzyme-containing preparation from Reference Example 1 (lyophilized whole cells or CFE, final concentration: 20 mg/mL) and an aqueous stock solution of D-glucose (9 mg/mL, final concentration: 50 mM). Thereto, a glucose dehydrogenase solution (final concentration: 2 mg/mL), a solution of 1,2-dehydroreticuline (14) (final concentration: 10 mM), KPi buffer (pH 7.5, final concentration: 0.1 M), NADP⁺ (20 mM stock solution, final concentration: 1 mM at the start of the reaction), and water were added to a final volume of 250 μL. The reaction mixtures were mixed at 30° C., 900 rpm, and quenched after 20 h by adding 250 μL of methanol. The samples were centrifuged, filtered through cotton wool, and analyzed by HPLC-UV. The measured values for yields and enantiomeric purity (enantiomeric excess, ee) of the four different approaches (cultivation in LB or TB medium; whole cells or CFE) are given in Table 1 below.

TABLE 1

| Medium | Praparation | Yield (%) | ee (%) |
|---|---|---|---|
| LB | whole cells | 97.6 | 98.3 |
| LB | CFE | 95.7 | 98.6 |
| TB | whole cells | 93.9 | 98.5 |
| TB | CFE | 99.9 | 98.7 |

Although all four enzyme variants delivered very good results, the best results were achieved with the cell-free extract of cells cultured in TB medium, which resulted in a virtually quantitative yield of (S)-reticuline with almost 99% enantiomeric purity. By optimizing the process conditions through routine experiments, it should be possible to even further improve these results to achieve an enantiomeric excess of >99.9%.

### Reference Example 2

Preparation of Dehydroreticuline Reductase from Papaver somniferum (PsDRR) (SEQ ID No: 1)
A) Transformation of Competent Arctic Express (DE3) E. coli Cells

A plasmid containing the Papaver somniferum gene encoding dehydroreticuline reductase (commercially purchased from BioCat GmbH and cloned into pET-47b, SEQ ID No: 2; 2 μL) and 100 μL of chemically competent Arctic Express (DE3) E. coli host cells were mixed in 1.5 mL microcentrifuge tubes. The mixtures were incubated on ice for 30 min. The competent cell/DNA mixtures were then shock-heated at 42° C. for 20 s. 700 μL of autoclaved LB medium were added to each sample, followed by shaking at 37° C., 300 rpm, for 1 h. Subsequently, 100 μL of the mixture were applied to previously prepared agar plates containing kanamycin, which were placed in an incubator at 37° C. overnight.

### B) Preparation of Overnight Cultures

50 mL plastic tubes were filled with 10 mL of LB medium, and 10 μL of kanamycin solution (50 mg/mL, final concentration: 50 μg/mL) were added. A single culture from each agar plate was taken for inoculation and cultured overnight at 30° C. and 120 rpm.

### C) Main Culture

For each batch, 330 mL of autoclaved LB medium were supplemented with 330 μL of kanamycin solution (50 mg/mL, final concentration: 50 μg/mL) and 3.3 mL of the respective overnight culture (1 mL per 100 mL of medium) were added. Cell growth proceeded at 30° C., 120 rpm, until the OD₆₀₀ value reached 1. The cultures were then induced with IPTG (1 M stock solution, final concentration: 1.0 mM). The cell cultures were shaken at 13-15° C., 120 rpm, for 20 to 24 hours.

To obtain the enzymes, the cultures were centrifuged (5000 rpm, 4° C., 20 min), resuspended in distilled water, and centrifuged again (5000 rpm, 4° C., 20 min). The pellets were resuspended in 10 mL of water per gram of wet pellet. The resuspensions were either sharp-frozen and lyophilized as whole cells or subjected to pulsed sonication for 5 min and centrifugation (14000 rpm, 4° C., 20 min). In the latter case, the pellets were discarded, and the supernatant was sharp-frozen and lyophilized. The resulting lyophilized cell-free extracts (CFE) and lyophilized whole cells were stored at −20° C.

### Example 8

### Preparation of (R)-reticuline (R)-(15)

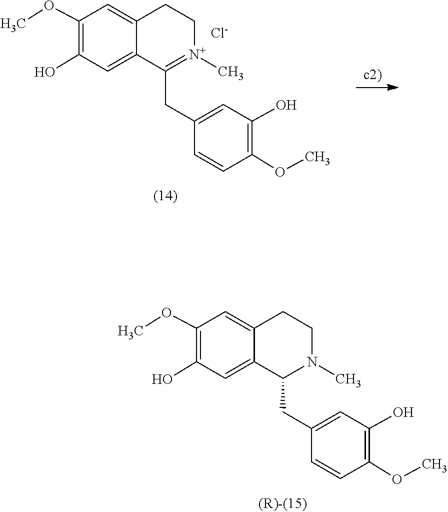

For this biotransformation, numerous approaches were carried out, each varying one of the reaction parameters. For example, the concentration of 1,2-dehydroreticuline (14) was varied between 10 and 50 mM, the pH was varied between 6 and 11 by selecting the respective buffer (KPi, Tris-HCl, or glycine-NaOH), and a water-miscible co-solvent, namely DMSO, EtOH, acetone, or isopropanol, was used, and its amount was varied between 2 and 30 vol %.

### Example 8A—Variation of Substrate Concentration

Microcentrifuge tubes (1.5 mL) were charged with an aqueous stock solution of D-glucose (9 mg/mL, final concentration: 50 mM) and the PsDRR enzyme-containing preparation from Reference Example 2 (lyophilized whole cells or CFE) in the form of a buffer stock solution having a concentration of 0.1 U/mL (final concentration: 0.06 U/mL). Thereto, a glucose dehydrogenase solution (final concentration: 2 mg/mL), different amounts of a solution of 1,2-dehydroreticuline (14) in a mixture of water and 15 vol % DMSO, so that the final concentration varied between 10 mM and 50 mM, NADP⁺ (20 mM stock solution, final concentration: 1 mM at the beginning of the reaction), Tris-HCl buffer (pH 8.5, final concentration: 0.1 M) and additional DMSO were added up to a final concentration of 10 vol %. The reaction mixtures were mixed at 30° C. and 900 rpm and quenched after 20 h by adding 400 μL of methanol. The samples were centrifuged, filtered through cotton wool, and analyzed by HPLC-UV. The yields for the respective substrate concentrations are given in Table 2 below.

TABLE 2

| | Concentration 1,2-dehydroreticuline | | | | | |
|---|---|---|---|---|---|---|
| | 0 mM | 10 mM | 20 mM | 30 mM | 40 mM | 50 mM |
| Yield (%) | 0 | 97.3 | 98.2 | 65.9 | 37.1 | 17.7 |

Thus, the best yield was achieved using a concentration of the starting product of 10-20 mM.

### Example 88—Variation of pH Value

Microcentrifuge tubes (1.5 mL) were loaded with an aqueous stock solution of D-glucose (9 mg/mL, final concentration: 50 mM) and a suspension of lyophilized whole cells containing the PsDRR enzyme in different buffers (KPi, Tris-HCl, or glycine-NaOH) at a concentration of 0.1 U/mL (final concentration: 0.06 U/mL). Thereto, a glucose dehydrogenase solution (final concentration: 2 mg/mL), a solution of 1,2-dehydroreticuline (14) in a mixture of water and 15 vol % DMSO (final concentration: 30 mM), NADP⁺ solution (final concentration: 1 mM at the start of the reaction), an appropriate amount of the respective buffer (0.1 M) to obtain the desired pH value and additional DMSO up to a final concentration of 10 vol % were added. The reaction mixtures were mixed at 30° C. and 900 rpm and quenched after 20 h by adding 250 μL of methanol. The samples were centrifuged, filtered through cotton wool, and analyzed by HPLC-UV. The yields for the respective pH and buffer are given in Table 3 below.

TABLE 3

| | pH | | | | | | | | | |
|---|---|---|---|---|---|---|---|---|---|---|
| | 6 | 6.5 | 7 | 7.5 | 8 | 8.5 | 9 | 9.5 | 10 | 10.5 |
| Buffer | Yield (%) | | | | | | | | | |
| KPi | 66.1 | 62.1 | 76.5 | 78.1 | 76.4 | | | | | |
| Tris-HCl | | | | | 99.3 | 98.2 | 76.0 | | | |
| glycine/NaOH | | | | | | | 81.3 | 99.0 | 99.3 | 43.5 |

Two maxima of the yields of (R)-reticuline are seen at pH 8-8.5 using Tris-HCl buffer and at 9.5-10 using glycine-NaOH buffer.

### Example 8C—Variation of Cosolvent

Microcentrifuge tubes (1.5 mL) were loaded with an aqueous stock solution of D-glucose (9 mg/mL, final concentration: 50 mM) and a suspension of lyophilized whole cells containing the PsDRR enzyme in water at a concentration of 0.1 U/m L (final concentration: 0.06 U/mL). Thereto, a glucose dehydrogenase solution (final concentration: 2 mg/mL), a solution of 1,2-dehydroreticuline (14) in water (concentration: 30 mM), varying amounts of a cosolvent (DMSO, EtOH, acetone or isopropanol) between 2 and 30 vol %, NADP⁺ solution (final concentration: 1 mM at the beginning of the reaction) and KPi buffer (pH 7.5, final concentration: 0.1 M) were added. The reaction mixtures were mixed at 30° C. and 900 rpm and quenched after 20 h by adding 250 μL of methanol. The samples were centrifuged, filtered through cotton wool, and analyzed by HPLC-UV. The yields for the respective amounts of cosolvent are given in Table 4 below.

TABLE 4

| | Concentration | |
|---|---|---|
| Cosolvent | (Vol./Vol.) | Yield (%) |
| DMSO | 0 | 33.6 |
| | 2 | 33.4 |
| | 5 | 38.6 |
| | 10 | 45.5 |
| | 15 | 32.4 |
| | 20 | 17.3 |
| | 25 | 6.0 |
| | 30 | 2.1 |
| EtOH | 2 | 32.2 |
| | 5 | 23.0 |
| | 10 | 4.8 |
| acetone | 2 | 33.2 |
| | 5 | 18.4 |
| | 10 | 3.9 |
| isopropanol | 2 | 31.7 |
| | 5 | 14.6 |
| | 10 | 2.6 |

It can be seen that the addition of 2 vol % of a cosolvent had almost no effect on the yields and that the latter could only be increased by the addition of DMSO, with the optimum appearing to be between 5 and 10 vol % or at about 10 vol % of DMSO in the aqueous solution.

### Example 8D—Variation of Reaction Time

In a further series of experiments using a combination of the best previously determined parameters, the reaction time was also varied, and it was found that when 1,2-dehydroreticuline (14) was used at a concentration of 10 mM, complete conversion was achieved after 4 h at the latest.

### Example 8E—Optimal Conditions

Thus, the best combination of reaction parameters for the enantioselective hydrogenation of 1,2-dehydroreticuline (14) to (R)-reticuline (R)-(15) appears to be that in which all of the following conditions i) to vii) apply:
i) 0.1 M Tris-HCl buffer or glycine-NaOH buffer is used as the solvent;
ii) 5-10 vol % or approximately 10 vol % DMSO are used as the cosolvent;
iii) the dehydroreticuline concentration is 10-20 mM;
iv) the pH is 8-8.5 (Tris-HCl) or 9-9.5 (glycine-NaOH);
v) NADPH/NADP⁺ is used as the cofactor at a concentration of 1 mM;
vi) glucose dehydrogenase is used as the coenzyme at a concentration of 2 mg/mL and D-glucose is used as the cosubstrate at a concentration of 9 mg/mL; and
vii) the reaction is allowed to proceed for 4 h (for 10 mM dehydroreticuline).

By selecting the following combination, (R)-reticuline was subsequently obtained in practically quantitative yield and almost enantiomerically pure form:
i) 0.1 M Tris-HCl buffer as solvent;
ii) 10 vol % DMSO as cosolvent;
iii) dehydroreticuline concentration 10 mM;
iv) pH 8;
v) 1 mM NADPH/NADP⁺;
vi) 2 mg/mL GDH and 9 mg/mL D-glucose; and
vii) 4 h reaction time.

When the enzyme PsDRR was used in the form of lyophilized whole cells, (R)-reticuline was obtained in a yield of 99.9% of theory, and when the cell-free extract was used in a yield of 97.5% of theory—in each case with an enantiomeric excess of >99.9% ee.

### Reference Example 3

### Preparation of the Corytuberine Synthase Enzyme

Both the wild type gene from Coptis japonica (SEQ ID No: 28), which encodes corytuberine synthase (CjCorSyn, SEQ ID No: 27), and a gene (SEQ ID No: 30), which encodes an N-terminally truncated version of the enzyme (Ncut-CorSyn, SEQ ID No: 29), were transformed into competent BL21(DE3) E. coli cells together with the gene (SEQ ID No: 6), which encodes the redox partner of corytuberine synthase, cytochrome P450 reductase 2 from Arabidopsis thaliana (AtR2, SEQ ID No: 5), which subsequently heterologously expressed the respective enzymes.

A1) Transformation of Competent BL21(DE3) E. coli Cells with the Reductase AtR2

The gene encoding the respective CorSyn from Coptis japonica and the gene encoding AtR2 from Arabidopsis thaliana were each purchased from BioCat GmbH in the form of the plasmid pET-28a. The AtR2 gene was cloned into the vector pCDF-1 b to enable co-expression of the two genes in two different plasmids. 2 μL of the solution containing the gene encoding AtR2 reductase from Arabidopsis thaliana (pCDF-1b) and 100 μL of chemically competent BL21(DE3) E. coli host cells were mixed in 1.5 mL microcentrifuge tubes. The mixtures were incubated on ice for 30 min. The competent cell/DNA mixtures were then shock-heated at 42° C. for 10 s. 700 μL of autoclaved LB medium were added to each sample, followed by shaking at 37° C., 300 rpm, for 1 h. Subsequently, 150 μL of the mixture were applied to previously prepared agar plates containing spectinomycin, which were placed in a 37° C. incubator overnight.

### A2) Preparation of Overnight Cultures

50 mL plastic tubes were filled with 10 mL of LB medium, and 50 μg/mL of spectinomycin solution were added. A single culture from each agar plate was taken for inoculation and cultured overnight at 37° C., 120 rpm.

### A3) Preparation of Electrokompetent Cells

500 mL of autoclaved LB medium were treated with 500 μL of spectinomycin solution (100 mg/mL, final concentration: 100 μg/mL) and 5.0 mL of the above overnight culture (1 mL per 100 mL medium) were added. Cell growth proceeded at 37° C., 120 rpm, until the OD₆₀₀ value was 0.5-0.7. The cells were chilled cooled for 20 min and transferred to sterile centrifuge cups. All subsequent steps were performed under sterile conditions, preferably at 0° C. (on ice).

To obtain the enzymes, the cultures were centrifuged (5000 rpm, 4° C., 15 min), the supernatant was discarded, and the cell pellet was carefully resuspended in 500 mL of 10% aqueous glycerol solution. The cells were centrifuged again (5000 rpm, 4° C., 15 min), and the cell pellet was resuspended in 250 mL of 10% aqueous glycerol solution. The cells were centrifuged again (5000 rpm, 4° C., 15 min), and the cell pellet was resuspended in 20 mL of 10% aqueous glycerol solution. Subsequently, the cell pellet was centrifuged for the last time (5000 rpm, 4° C., 15 min) and resuspended in 2 mL of 10% aqueous glycerol solution, aliquoted in 80 μL portions, sharp-frozen, and stored at −80° C.

A4) Transformation of the Electrokompetent Cells with CorSyn

A plasmid containing the gene encoding the respective CorSyn (pET-28a, 2 μL) and 80 μL of electrocompetent BL21(DE3) E. coli host cells containing the AtR2 plasmid (in pCDF-1 b) were mixed in 1.5 mL microcentrifuge tubes and incubated for 1 min. The respective cell suspension was transferred to an electroporation cuvette, where the cells settled to the bottom. The cuvette was placed in the electroporation chamber and subjected to electropulses using a MicroPulser electroporator (BioRad) (program: Ec2). The cuvette was then removed from the chamber, and to its content 1 mL of SOC medium were added and gently mixed. The cell suspension was transferred to 1.5 mL microcentrifuge tubes, followed by shaking at 37° C., 300 rpm, for 1 h. Subsequently, 100 μL of the mixture were applied to previously prepared agar plates containing kanamycin and spectinomycin, which were placed in a 37° C. incubator overnight.

### B) Preparation of Overnight Cultures

50 mL plastic tubes were filled with 10 mL of LB medium, and 10 μL each of kanamycin solution (50 mg/mL, final concentration: 50 μg/mL) and spectinomycin solution (100 mg/mL, final concentration: 100 μg/mL) were added. A single culture from each agar plate was taken for inoculation and cultured overnight at 37° C. and 120 rpm.

### C) Main Culture and Recovery of the Enzymes

5 mL of TB medium were supplemented with 5 μL of kanamycin solution and 5 μL of spectinomycin solution (from 50 and 100 mg/mL stock solutions, final concentration: 50 or 100 μg/mL, respectively) and inoculated with 1 vol % of the overnight cultures in 10 mL deep well plates. Cells were grown at 20° C., 120 rpm, for 9 h. Cultures were induced with 5 μL of IPTG (1 mM) supplemented with 5 μL of 5-aminolevulinic acid (from a 1 M stock solution, final concentration: 1 mM) and incubated at 20° C., 120 rpm, for 22 h. The cultures were centrifuged (4000 rpm, 15° C., 30 min) and the pellet was used for biotransformations.

### Example 9

### Preparation of Corytuberine (16)

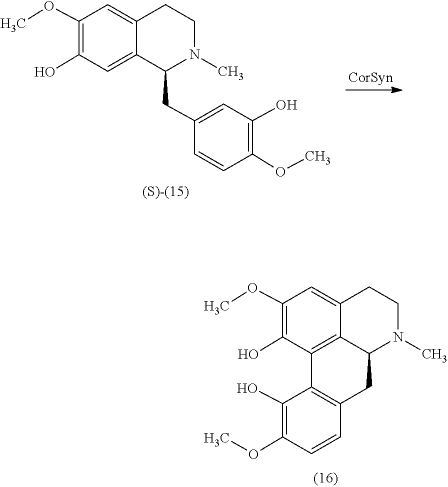

The pellets obtained in Reference Example 3 were resuspended in 0.5 mL of KPi buffer (0.1 M, pH 7.5) and transferred to 2 mL deep well plates, after which the substrate, (S)-reticuline (S)-(15) (from a racemic 100 mM stock solution in DMSO, final concentration: 0.2 mM rac-reticuline), NADPH solution (from 25 mM stock solution, final concentration: 0.05 mM), and D-glucose solution (from 1.25 M stock solution, final concentration: 50 mM) were added. The reaction mixtures were incubated at 30° C., 220 rpm, for 20 h. The samples were quenched with 200 μL of methanol and transferred to fresh microcentrifuge tubes. The samples were centrifuged (14,680 rpm, 10 min), and the supernatant was filtered through cotton wool. The samples were analyzed qualitatively by TLC and quantitatively by HPLC-MS.

When using host cells transformed with the wild type gene from Coptis japonica (CjCorSyn), corytuberine was obtained in a yield of 14% of theory, whereas a yield of 45% of theory was observed for BL21(DE3) cells transformed with the N-terminally truncated version of the gene (NcutCorSyn). Thus, the use of NcutCorSyn as the enzyme for producing corytuberine from (S)-reticuline is clearly preferable. Without wishing to be bound by a specific theory, the inventors assume that this higher efficacy is due to a stronger expression of the truncated enzyme in the host cells.

### Reference Example 4

### Preparation of the Salutaridine Synthase Enzyme

The wild type gene from Papaver somniferum (SEQ ID No: 8), which codes for the salutaridine synthase (PsSalSyn, SEQ ID No: 7), as well as nine other genes (SEQ ID Nos: 10, 12, 14, 16, 18, 20, 22, 24, 26), which code for various mutated or modified versions of the enzyme (SEQ ID Nos: 9, 11, 13, 15, 17, 19, 21, 23, 25), were analyzed, each together with the gene (SEQ ID No: 6), which codes for the redox partner of the salutaridine synthase, the cytochrome P450 reductase 2 from Arabidopsis thaliana. (AtR2, SEQ ID No: 5), were transformed into competent BL21(DE3) or Arctic Express (DE3) E. coli cells, which subsequently heterologously expressed the respective enzymes.

The nine other versions of the enzyme besides the wild type include an N-terminally truncated version of the enzyme (NcutSalSyn, SEQ ID No: 9), a chimeric version of it that includes a peptide sequence of another cytochrome P450 enzyme, namely cheilanthifoline synthase CYP719A5, at the N-terminal end (CFS_PsSalSyn, SEQ ID No: 11), a version each supplemented with glutathione S-transferase or small ubiquitin-like modifier tag of both the wild type (GST-tag PsSalSyn, SEQ ID No: 13; SUMO-tag PsSalSyn, SEQ ID No: 17) and the N-terminally truncated version (GST-tag NcutSalSyn, SEQ ID No: 15; SUMO-tag NcutSalSyn, SEQ ID No: 19), as well as three constructs in which the highly hydrophobic native part at the N-terminal end was exchanged for 6 to 8 amino acids (NswapAL_SalSyn, SEQ ID No: 21; NswapAK_SalSyn, SEQ ID No: 23; NswapKK_SalSyn, SEQ ID No: 25). Three of these genes (wild type, SEQ ID No: 8; NcutSalSyn, SEQ ID No: 10; and CFS_PsSalSyn, SEQ ID No: 12) were each purchased from BioCat GmbH in the form of the plasmid pET-28a(+). The wild type gene and the gene encoding NcutSalSyn were cloned, using restriction cloning, into the vectors pDB-HisGST and pCIOX which provided the respective genes with GST or SUMO tags. The three constructs in which the N-terminal part of the protein was modified (Nswap-AL_SalSyn, Nswap-AK_SalSyn, and Nswap-KK_SalSyn) were prepared using PCR with appropriate primers.

A1) Transformation of Competent BL21(DE3) or Arctic Express (DE3) E. coli Cells

The plasmid pCDF-1b (2 μL) containing the gene encoding the cytochrome P450 reductase AtR2 and 100 μL of chemically competent BL21(DE3) or Arctic Express (DE3) E. coli host cells were mixed in 1.5 mL microcentrifuge tubes. The mixtures were incubated on ice for 30 min. The competent cell/DNA mixtures were then shock-heated at 42° C. for 10 s (BL21) or 20 s (Arctic Express). 700 μL of autoclaved LB medium were added to each sample, followed by shaking at 37° C. at 300 rpm for 1 h. Subsequently, 150 μL of the mixture were applied to previously prepared agar plates containing spectinomycin, which were placed in a 37° C. incubator overnight.

### A2) Preparation of Overnight Cultures

50 mL plastic tubes were filled with 10 mL of LB medium, and 50 μg/mL of spectinomycin solution were added. A single culture from each agar plate was taken for inoculation and cultured overnight at 37° C. and 120 rpm.

### A3) Preparation of Electrocompetent Cells

500 mL of autoclaved LB medium were supplemented with 500 μL of spectinomycin solution (100 mg/mL, final concentration: 100 μg/mL) and 5.0 mL of the above overnight culture of BL21(DE3) or Arctic Express(DE3) E. coli cells (1 mL per 100 mL of medium) were added. Cell growth proceeded at 37° C., 120 rpm, until the OD₆₀₀ value was 0.5-0.7. The cells were cooled on ice for 20 min and transferred to sterile centrifuge cups. All subsequent steps were performed under sterile conditions, preferably at 0° C. (on ice).

To obtain the enzymes, the cultures were centrifuged (5000 rpm, 4° C., 15 min), the supernatant was discarded, and the cell pellet was carefully resuspended in 500 mL of 10% aqueous glycerol solution. The cells were centrifuged again (5000 rpm, 4° C., 15 min), and the cell pellet was resuspended in 250 mL of 10% aqueous glycerol solution. The cells were centrifuged again (5000 rpm, 4° C., 15 min), and the cell pellet was resuspended in 20 mL of 10% aqueous glycerol solution. Subsequently, the cell pellet was centrifuged for the last time (5000 rpm, 4° C., 15 min) and resuspended in 2 mL of 10% aqueous glycerol solution, aliquoted into 80 μL portions, sharp-frozen, and stored at −80° C.

A4) Transformation of the Electrocompetent Cells with SalSyn

The plasmid pET-28a (2 μL) containing the respective SalSyn gene and 80 μL of electrocompetent BL21(DE3) or Arctic Express (DE3) E. coli cells containing the AtR2 plasmid (in pCDF-1 b) were mixed in 1.5 mL microcentrifuge tubes and incubated for 1 min. The respective cell suspension was transferred to an electroporation cuvette, where the cells settled to the bottom. The cuvette was placed in the electroporation chamber and subjected to electropulses using a MicroPulser electroporator (BioRad) (program: Ec2). The cuvette was then removed from the chamber, and to its content 1 mL of SOC medium were added and gently mixed. The cell suspension was transferred to 1.5 mL microcentrifuge tubes, followed by shaking at 37° C., 300 rpm, for 1 h. Subsequently, 100 μL of the mixture was applied to previously prepared agar plates containing kanamycin and spectinomycin, which were placed in a 37° C. incubator overnight.

### B) Preparation of Overnight Cultures

50 mL plastic tubes were filled with 10 mL of LB medium, and 50 μg/mL kanamycin and 100 μg/mL spectinomycin were added. A single culture from each agar plate was taken for inoculation and cultured overnight at 37° C., 120 rpm.

### C) Main Culture and Recovery of the Enzymes

5 mL of TB medium were supplemented with 5 μL of kanamycin solution and 5 μL of spectinomycin solution (from 50 and 100 mg/mL stock solutions, final concentration: 50 or 100 μg/mL, respectively) and inoculated with 1 vol % of the overnight cultures in 10 mL deep well plates. Depending on the approach and the host cells, BL21(DE3) or Arctic Express (DE3) E. coli cells, the cells were incubated at 20° C., 30° C., or 37° C., at 120 rpm, for 9 or 6 hours, respectively. Cultures were induced with 5 μL of IPTG (1 mM) supplemented with 5 μL of 5-aminolevulinic acid (from a 1 M stock solution, final concentration: 1 mM) and incubated at 20° C., 120 rpm, for 22 h. The cultures were centrifuged (4000 rpm, 15° C., 30 min) and the pellet was used for biotransformations.

### Example 10

### Preparation of Salutaridine (17)

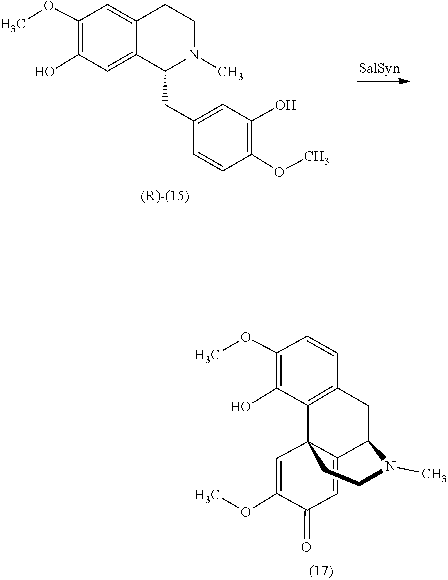

The pellets obtained in Reference Example 4 were resuspended in 0.5 mL of KPi buffer (0.1 M, pH 7.5) and transferred to 2 mL deep-well plates, after which the substrate (R)-reticulin (R)-(15) (from a racemic 100 mM stock solution in DMSO, final concentration: 0.2 mM rac-reticulin), NADPH solution (from 25 mM stock solution, final concentration: 0.05 mM), and D-glucose solution (from 1.25 M stock solution, final concentration: 50 mM) were added. The reaction mixtures were incubated at 3000, 220 rpm, for 20 h. The samples were quenched with 200 μL of methanol and transferred to fresh microcentrifuge tubes. The samples were centrifuged (14,680 rpm, 10 min), and the supernatant was filtered through cotton wool. The samples were analyzed qualitatively by HPLC-MS, HR-MS, and TLC, and quantitatively by HPLC-UV.

The yield results for the ten different enzyme combinations produced in the respective E. coli host cell cultures, BL21 (DE3) (“BL21”) or Arctic Express (DE3) (“AE”) are given in Table 5 below along with the incubation temperature and duration for cell proliferation and expression, respectively.

TABLE 5

| | | Cell | | | | |
|---|---|---|---|---|---|---|
| | | proliferation | | Expression | | |
| Host | | temp. | time | temp. | time | Yield |
| cell | Enzyme | (° C.) | (h) | (° C.) | (h) | (%) |
| BL21 | NcutSalSyn, | 20 | 9 | 20 | 22 | 43.4 |
| | SEQ ID No: 9 | 37 | 6 | 20 | 22 | 17 |
| BL21 | PsSalSyn, | 20 | 9 | 20 | 22 | 58.1 |
| | SEQ ID No: 7 | 37 | 6 | 20 | 22 | 0 |
| BL21 | CFS_PsSalSyn, | 20 | 9 | 20 | 22 | 85.2 |
| | SEQ ID No: 11 | 37 | 6 | 20 | 22 | 98 |
| AE | GST-tag | 20 | 9 | 13 | 22 | 29.8 |
| | NcutSalSyn | 30 | 6 | 13 | 22 | 2 |
| | SEQ ID No: 15 | | | | | |
| AE | GST-tag | 20 | 9 | 13 | 22 | 6.3 |
| | PsSalSyn, | 30 | 6 | 13 | 22 | 4 |
| | SEQ ID No: 13 | | | | | |
| AE | SUMO-tag | 20 | 9 | 13 | 22 | 82.8 |
| | NcutSalSyn, | 30 | 6 | 13 | 22 | 26 |
| | SEQ ID No: 19 | | | | | |
| AE | SUMO-tag | 20 | 9 | 13 | 22 | 9.9 |
| | PsSalSyn, | 30 | 6 | 13 | 22 | 4 |
| | SEQ ID No: 17 | | | | | |
| BL21 | NswapAL_SalSyn, | 20 | 9 | 20 | 22 | 0.0 |
| | SEQ ID No: 21 | 37 | 6 | 20 | 22 | 3 |
| BL21 | NswapAK_SalSyn, | 20 | 9 | 20 | 22 | 28.2 |
| | SEQ ID No: 23 | 37 | 6 | 20 | 22 | 16 |
| BL21 | NswapKK_SalSyn, | 20 | 9 | 20 | 22 | 100 |
| | SEQ ID No: 25 | 37 | 6 | 20 | 22 | 65 |

It can be seen that the efficacy of the respective salutaridine synthase varies greatly depending on the host cell and the expression and cultivation conditions. Nevertheless, the table also clearly shows that with appropriate choice of conditions, salutaridine could be obtained in quantitative yield, with the enzymes with the best yields being CFS_PsSalSyn and NswapKK_SalSyn.

The present invention thus provides a process by which the alkaloids corytuberine and salutaridine can be obtained in very good yields by means of fewer reaction steps, which was not possible according to prior art to date.

1. A method for preparing corytuberine and salutaridine via the benzylisoquinoline alkaloid reticuline as an intermediate, the process comprising the following reaction steps:
a) amidation of 2-(4-hydroxy-3-methoxyphenyl)-N-methylethanamine (12) with 3-hydroxy-4-methoxyphenylacetic acid to yield N-[2-(4-hydroxy-3-methoxyphenyl)-ethyl-N-methyl-3-hydroxy-4-methoxyphenylacetic acid amide (13): 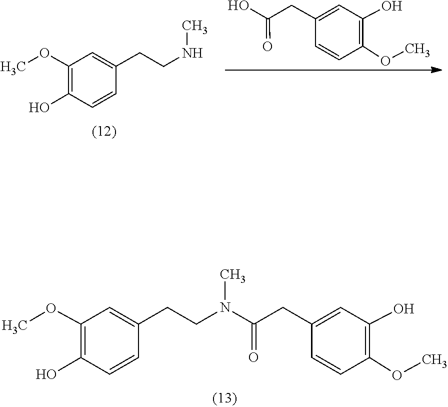
b) Bischler-Napieralski cyclization of the tertiary amide (13) to yield 1,2-dehydro-reticuline (14): 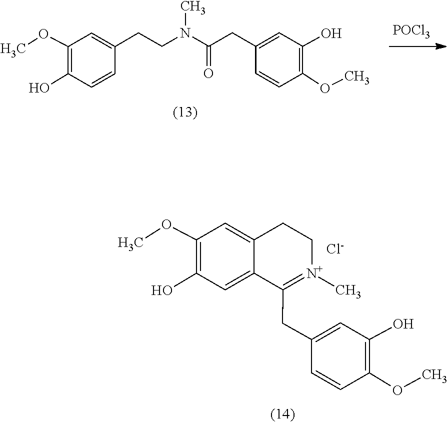
c) biocatalytic enantioselective hydrogenation of 1,2-dehydroreticuline (14) to yield reticuline (15), either
c1) using an imine reductase to yield (S)-reticuline (S)-(15) or
c2) using a dehydroreticuline reductase to yield (R)-reticuline (R)-(15): 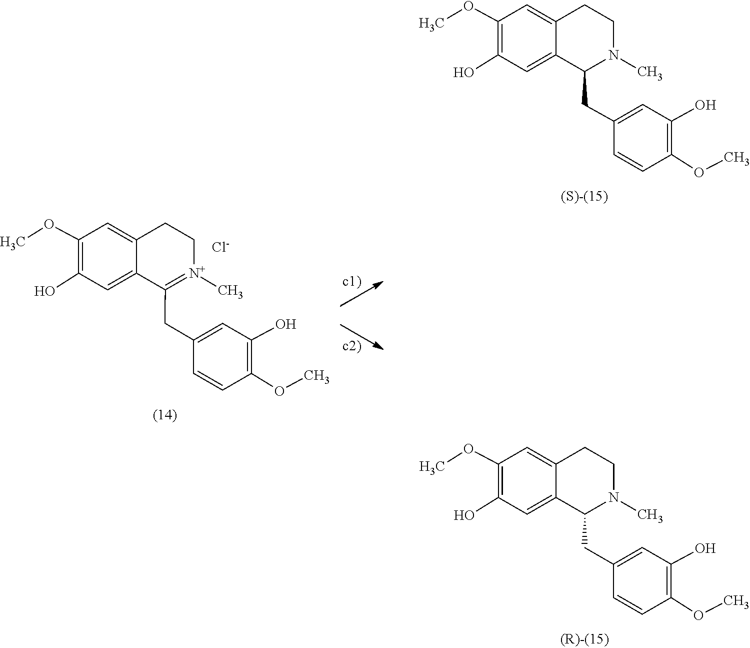
d) biocatalytic oxidative phenol coupling between the two aromatic rings of the respective reticuline, namely either
d1) ortho-ortho coupling of (S)-reticuline in the presence of corytuberine synthase (CorSyn) to yield corytuberine (16) or
d2) ortho-para coupling of (R)-reticuline in the presence of salutaridine synthase (SalSyn) to yield salutaridine (17):

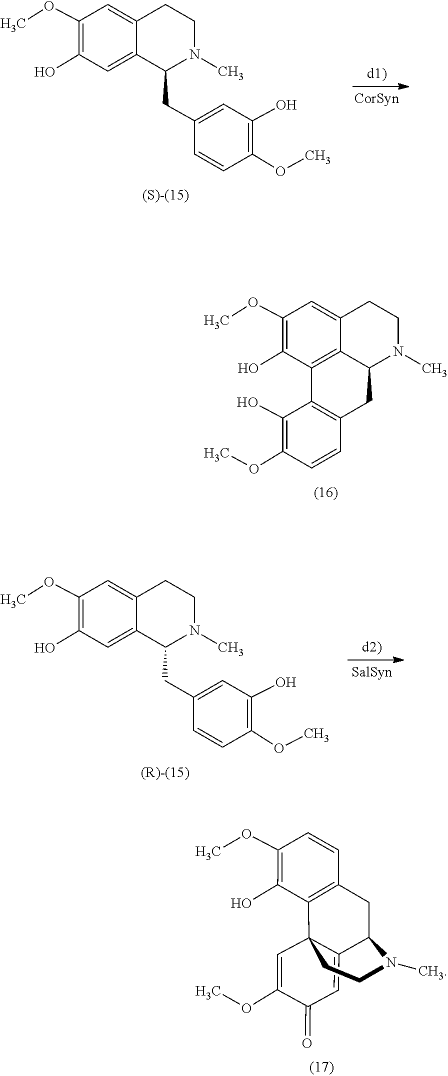

2. The method according to claim 1, wherein said amidation in step a) is carried out using a carbodiimide,
optionally wherein 1-ethyl-3-(3-dimethylaminopropyl)carbodiimide (EDC) is used as said carbodiimide,
optionally wherein the amidation is carried out in the presence of hydroxyiminocyanoacetic acid ethyl ester (Oxyma) as an additive.

3. The method according to claim 1, wherein said Bischler-Napieralski cyclization in step b) is carried out in acetonitrile as a solvent, optionally under reflux.

4. The method according to claim 1, wherein said biocatalytic enantioselective hydrogenation in step c1) is carried out using the mutant imine reductase IR45 F190M-W191F,
optionally wherein a recombinant enzyme heterologously expressed by E. coli cells, optionally by competent BL21(DE3) cells, is used in the form of the lyophilized cells or a cell-free extract thereof.

5. The method according to claim 4, wherein the hydrogenation in step c1) is carried out using one or more or all of the following conditions i) to v):
i) 0.1 M KPi buffer is used as a solvent;
ii) the dehydroreticuline concentration is 10-30 mM, optionally 10-20 mM, optionally 10 mM;
iii) the pH is 7-8.5, optionally about 7.5;
iv) NADPH/NADP⁺ is used as a cofactor, optionally at a concentration of 1 mM;
v) glucose dehydrogenase is used as a coenzyme, optionally at a concentration of 2 mg/mL, and D-glucose is used as a cosubstrate, optionally at a concentration of 9 mg/mL.

6. The method according to claim 1, wherein said biocatalytic enantioselective hydrogenation in step c2) is carried out using the dehydroreticuline reductase from Papaver somniferum (PsDRR),
optionally wherein a recombinant enzyme heterologously expressed by E. coli cells, optionally by competent Arctic Express (DE3) cells, is used in the form of the lyophilized cells or a cell-free extract thereof.

7. The method according to claim 6, wherein said biocatalytic enantioselective hydrogenation in step c2) is carried out using one or more or all of the following conditions i) to vi):
i) 0.1 M Tris-HCl buffer or glycine-NaOH buffer is used as a solvent;
ii) 5-10 vol % DMSO are used as a cosolvent;
iii) the dehydroreticuline concentration is 10-30 mM, optionally 10-20 mM;
iv) the pH is 8-8.5 or 9-9.5;
v) NADPH/NADP⁺ is used as a cofactor, optionally at a concentration of 1 mM;
vi) glucose dehydrogenase is used as a coenzyme, optionally at a concentration of 2 mg/mL, and D-glucose is used as a cosubstrate, optionally at a concentration of 9 mg/mL.

8. The method according to claim 1, wherein said biocatalytic oxidative phenol coupling between the two aromatic rings of (S)-reticuline in step d1) is carried out using a corytuberine synthase, optionally the corytuberine synthase from Coptis japonica (CjCorSyn) or a modified version thereof, together with a cytochrome P450 reductase, optionally the cytochrome P450 reductase 2 from Arabidopsis thaliana (AtR2) as a redox partner,
optionally wherein the corytuberine synthase and AtR2 are used as recombinant enzymes heterologously expressed by E. coli cells, optionally by competent BL21(DE3) cells, in the form of whole cells or a cell-free extract thereof.

9. The method according to claim 8, wherein said ortho-ortho phenol coupling in step d1) is carried out using one or more or all of the following conditions i) to vii):
i) 0.1 M KPi buffer is used as a solvent;
ii) the enzymes are used in the form of resuspended whole cells;
iii) 0.2-10 vol % DMSO are used as a cosolvent;
iv) the reticuline concentration is 0.1-5 mM;
v) the pH is 7.5-8.0;
vi) NADPH/NADP⁺ is used as a cofactor, optionally at a concentration of 0.05 mM;
vii) a glucose dehydrogenase preparation is used as a coenzyme, optionally at a concentration of 2 mg/mL, and D-glucose is used as a cosubstrate, optionally at a concentration of 50 mM.

10. The method according to claim 1, wherein said biocatalytic oxidative phenol coupling between the two aromatic rings of (R)-reticuline in step d2) is carried out using a salutaridine synthase, optionally the salutaridine synthase from Papaver somniferum (PsSalSyn), a chimeric version thereof or a construct thereof (Nswap_KK_SalSyn), together with a cytochrome P450 reductase, optionally the cytochrome P450 reductase 2 from Arabidopsis thaliana (AtR2) as a redox partner,
optionally wherein the salutaridine synthase and AtR2 are used as recombinant enzymes heterologously expressed by E. coli cells, optionally by competent BL21(DE3) cells, in the form of the whole cells or a cell-free extract thereof.

11. The method according to claim 10, wherein said ortho-para coupling of von (R)-reticuline in step d2) is carried out using one or more or all of the following conditions i) to vii):
i) 0.1 M KPi buffer is used as a solvent;
ii) the enzymes are used in the form of resuspended whole cells;
iii) 0.2-2 vol % DMSO are used as a cosolvent;
iv) the reticuline concentration is 0.1-0.5 mM;
v) the pH is 7.5-8.0;
vi) NADPH/NADP⁺ is used as a cofactor, optionally at a concentration of 0.05 mM;
vii) a glucose dehydrogenase preparation is used as a coenzyme, optionally at a concentration of 2 mg/mL, and D-glucose is used as a cosubstrate, optionally at a concentration of 50 mM.

12. The method according to claim 1, wherein the starting product 2-(4-hydroxy-3-methoxyphenyl)-N-methyl-ethanamine (12) used in step a) is previously prepared starting from 4-allyl-2-methoxyphenol (eugenol) by the following reaction steps:
I) ozonolysis of 4-allyl-2-methoxyphenol (9) to yield 4-hydroxy-3-methoxy-phenylacetaldehyde (homovanillin) (10): 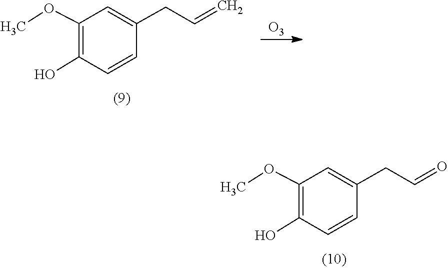
II) reductive amination of the aldehyde (10) with N-benzylmethanamine to yield the benzyl-protected intermediate N-benzyl-2-(4-hydroxy-3-methoxyphenyl)-N-methylethanamine (11): 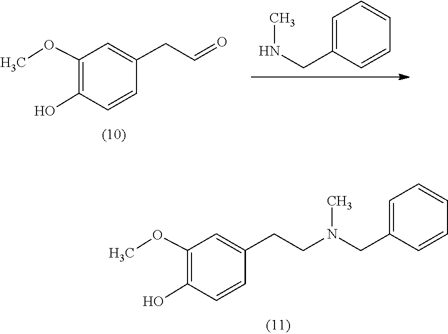
III) hydrogenolysis to remove the benzyl-protecting group to obtain the desired amine (12):

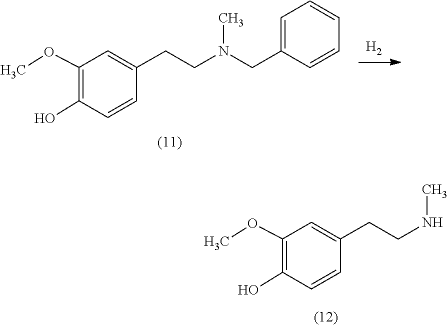

13. The method according to claim 12, wherein
the ozonolysis of eugenol (9) in step I) is carried out in an organic solution by introducing a mixture of O₃/O₂ at a temperature <0° C., optionally at −15° C., and subsequently quenching with dimethyl sulfide; and/or
the reductive amination of the aldehyde (10) in step II) is carried out in an organic solution in the presence of a borohydride, optionally NaBH(OAc)₃, with ice bath cooling; and/or
the hydrogenolysis in step III) is carried out in an alcoholic solution in the presence of a palladium catalyst, optionally Pd/C, and HCl at room temperature.

14. The method according to claim 12, wherein the steps I) and II) are carried out as a one-pot reaction.
